SEQUENCE LISTING (29 sequences)

SEQ ID NO: 1 - DNA, 32 bp, Artificial Sequence
  primer
aattactagtgggacagtactgaagactctgc

SEQ ID NO: 2 - DNA, 34 bp, Artificial Sequence
  primer
aattaagcttagtgtacaaactttattgaaacgc

SEQ ID NO: 3 - DNA, 25 bp, Artificial Sequence
  primer
caggcctccctaattctccctggac

SEQ ID NO: 4 - DNA, 25 bp, Artificial Sequence
  primer
gtccagggagaattagggaggcctg

SEQ ID NO: 5 - RNA, 23 nt, Homo sapiens
gcagcaccucccccuaaccaaau

SEQ ID NO: 6 - RNA, 21 nt, Homo sapiens
cuugguucagggaggguccca

SEQ ID NO: 7 - RNA, 23 nt, Homo sapiens
gcagcaccucucccuaaccaaau

SEQ ID NO: 8 - RNA, 21 nt, Homo sapiens
cuugguucagggaggguccca

SEQ ID NO: 9 - DNA, 41 bp, Homo sapiens
aggcctccctagcacctccccctaaccaaattctccctgga

SEQ ID NO: 10 - DNA, 41 bp, Homo sapiens
aggcctccctagcacctctccctaaccaaattctccctgga

SEQ ID NO: 11 - DNA, 23 bp, Homo sapiens
aggcctccctaattctccctgga

SEQ ID NO: 12 - DNA, 19 bp, Artificial Sequence
  primer
gagcgagccagctcagtag

SEQ ID NO: 13 - DNA, 21 bp, Artificial Sequence
  primer
gctcccaaagcgattctccta

SEQ ID NO: 14 - DNA, 19 bp, Artificial Sequence
  primer
ctgggattacaaatgtgag

SEQ ID NO: 15 - DNA, 19 bp, Artificial Sequence
  primer
ctgggattacaagtgtgag

SEQ ID NO: 16 - DNA, 21 bp, Artificial Sequence
  primer
ctgcacagatcagacccacaa

SEQ ID NO: 17 - DNA, 20 bp, Artificial Sequence
  primer
cactgggcaggcttatgaga

SEQ ID NO: 18 - DNA, 17 bp, Artificial Sequence
  primer
tcaggaagacgtgattt

SEQ ID NO: 19 - DNA, 17 bp, Artificial Sequence
  primer
tcaggaagacctgattt

SEQ ID NO: 20 - DNA, 20 bp, Artificial Sequence
  primer
acggacctcctcactaagct

SEQ ID NO: 21 - DNA, 18 bp, Artificial Sequence
  primer
gccccacccctgtatctg

SEQ ID NO: 22 - DNA, 16 bp, Artificial Sequence
  primer
acaggtaccagaggca

SEQ ID NO: 23 - DNA, 14 bp, Artificial Sequence
  primer
aggtaccaaaggca

SEQ ID NO: 24 - DNA, 23 bp, Artificial Sequence
  primer
tagtgtcaccctcaaaccccagt

SEQ ID NO: 25 - DNA, 23 bp, Artificial Sequence
  primer
ccacggagttgttacctgtggac

SEQ ID NO: 26 - DNA, 16 bp, Artificial Sequence
  primer
gcgcaatctcaggtca

SEQ ID NO: 27 - DNA, 17 bp, Artificial Sequence
  primer
accaccgtgtctggcta

SEQ ID NO: 28 - DNA, 22 bp, Artificial Sequence
  primer
tcccatttctcccttctagttg

SEQ ID NO: 29 - DNA, 19 bp, Artificial Sequence
  primer
aagttgaggctgcagggtg

(19) United States
(12) Patent Application Publication
(10) Pub. No.: US 2009/0291444 A1
(43) Pub. Date: Nov. 26, 2009

(54) METHODS AND MATERIALS FOR DETECTING AND TREATING DEMENTIA

(76) Inventors: Jason Eriksen, Houston, TX (US); Dennis W. Dickson, Jacksonville, FL (US); Matthew C. Baker, Jacksonville, FL (US); Rosa Rademakers, Ponte Vedra Beach, FL (US); Todd Robinson, Kirkland, WA (US)

Correspondence Address: FISH & RICHARDSON P.C., PO BOX 1022, MINNEAPOLIS, MN, 55440-1022, US

(21) Appl. No.: 12/413,869

(22) Filed: Mar. 30, 2009

Related U.S. Application Data
(60) Provisional application No. 61/041,058, filed on Mar. 31, 2008.

(51) Int. Cl. C12Q 1/68

(52) U.S. Cl. 435/6

(57) ABSTRACT

This document relates to methods and materials involved in detecting mutations linked to dementia (e.g., frontotemporal lobar degeneration). For example, methods and materials for determining whether or not a mammal is homozygous for a mutant T allele of rs5848 are provided. This document also relates to methods and materials involved in treating mammals having or being susceptible to developing neurodegenerative disorders (e.g., frontotemporal lobar degeneration). For example, methods and materials for inhibiting the ability of miRNA to suppress GRN polypeptide expression in mammals are provided.

### CROSS REFERENCE TO RELATED PATENTS

This document claims priority to U.S. Ser. No. 61/041,058, filed Mar. 31, 2008.

### STATEMENT AS TO FEDERALLY SPONSORED RESEARCH

This invention was made with government support under grant P50AG16574 awarded by The National Institute of Aging. The government has certain rights in the invention.

### BACKGROUND

1. Technical Field

This document relates to methods and materials involved in detecting mutations linked to dementia (e.g., frontotemporal lobar degeneration). This document also relates to methods and materials involved in treating mammals having or being susceptible to developing neurodegenerative disorders (e.g., frontotemporal lobar degeneration).

2. Background Information

Frontotemporal lobar degeneration (FTLD) is a progressive neurodegenerative disorder representing about 5 percent of all dementia patients (Graff-Radford and Woodruff, Semin. Neurol., 27:48-57 (2007)). It is the second most common form of early-onset neurodegenerative dementia after Alzheimer's Disease (AD), affecting 10-20 percent of patients with an onset of dementia before 65 years. FTLD patients present with prominent behavioral and personality changes, often accompanied by language impairment, which evolve gradually into cognitive impairment and dementia (McKhann et al., Arch. Neurol., 58:1803-1809 (2001) and Neary et al., Neurology, 51:1546-54 (1998)). FTLD may occur alone or in combination with motor neuron disease (MND) (Lomen-Hoerth et al., Neurology, 59:1077-79 (2002)). The most common neuropathology associated with clinical FTLD is frontal and anterior temporal lobe atrophy with neuronal inclusions immunoreactive for ubiquitin and TAR-DNA binding protein 43 (TDP-43), but negative for tau and α-synuclein (FTLD-U) (Josephs et al., Neuropathol. Appl. Neurobiol., 30:369-73 (2004); Lipton et al., Acta. Neuropathol. (Berl), 108:379-85 (2004); and Mackenzie et al., Acta. Neuropathol., 112:551-59 (2006)). Neuronal cytoplasmic inclusions (NCIs) in the neocortex, striatum, and the dentate fascia of the hippocampus are the pathological hallmarks of FTLD-U. Up to four subtypes of FTLD-U have been delineated that are based on the distribution of NCIs, dystrophic neurites and the presence of neuronal intranuclear inclusions (NIIs). Almost all cases with PGRN mutations have a common FTLD-U subtype, characterized by NCIs, short thin neurites in layer II of the cortex and lentiform NIIs. This subtype is referred to as Type 1 by Mackenzie and coworkers (Mackenzie et al., Acta. Neuropathol., 112:539-49 (2006)) and Type 3 by Sampathu and co-workers (Sampathu et al., Am. J. Pathol., 169:1343-52 (2006)).

FTLD has a high familial incidence, with up to 50% of patients reported to have a family history of dementia. Recent molecular genetic advances in the field of FTLD have revealed that the genetic basis of FTLD-U is heterogeneous, and the causative mechanisms are just starting to be unraveled (Rademakers and Hutton, Curr. Neurol. Neurosci. Rep., 7:434-42 (2007)). Loss-of-function mutations in the gene encoding the secreted growth factor progranulin (PGRN) on chromosome 17 were identified as a major cause of familial FTLD-U, and are present in up to 25 percent of familial FTLD-U patients worldwide (Baker et al., Nature, 442:916-9 (2006); Cruts et al., Nature, 442:920-4 (2006); and Gass et al., Hum. Mol. Genet., 15:2988-3001 (2006)). In addition, mutations in the valosin containing protein gene (VCP) and the gene encoding the charged multivesicular body protein (CHMP2B) were reported in a small number of FTLD-U families (Skibinski et al., Nat. Genet., 37:806-8 (2005) and Watts et al., Nat. Genet., 36:377-81 (2004)).

### SUMMARY

This document relates to methods and materials for detecting mutations that are linked to dementia. The methods and materials provided herein are based, in part, on the discovery that mutations within progranulin (GRN) nucleic acid are linked to dementia (e.g., FTLD or AD). The human GRN gene is located at chromosome 17q21, and its coding sequence is available at GenBank® Accession Number M75161 (g.i.:183612). The GRN gene is also known as epithelin precursor, proepithelin, PEPI, acrogranin, and granulin. A GRN gene can have 12 exons that together can encode a polypeptide with a molecular weight of 68.5 kDa. Granulins form a family of cysteine-rich polypeptides, some of which have growth modulatory activity. The widespread occurrence of GRN mRNA in cells from the hematopoietic system and in epithelia implies functions in these tissues. At least four different human granulin polypeptides can be processed from a single GRN precursor which can contain 7.5 repeats that each contain 12 conserved cysteine residues. Both the GRN precursor and processed GRN polypeptides can have biological activity. The term “GRN polypeptide” as used herein includes, without limitation, human GRN polypeptides (e.g., human GRN polypeptides set forth in GenBank® under g.i. numbers 183612, 4504151, and 77416865). A human progranulin polypeptide can be a 593-amino acid glycosylated polypeptide having a consensus sequence that is repeated seven and a half times.

This document provides methods and materials for detecting GRN nucleic acid containing the mutant ‘T’ allele of rs5848. For example, standard PCR techniques can be used to amplify a fragment of a patient's GRN nucleic acid. The amplified fragment can be sequenced or probed using standard techniques to determine whether or not the fragment contains the mutant ‘T’ allele of rs5848. In some cases, a mammal found to be homozygous or heterozygous for the ‘T’ alleles of rs5848 can be classified as having or being susceptible to developing neurodegenerative disorders (e.g., FTLD or AD). Detecting such mutations can allow clinicians to assess patients for disease risk and plan treatment options for the patient.

This document also relates to methods and materials for treating a mammal having or being likely to develop a neurodegenerative disorder (e.g., FTLD or AD). For example, this document relates to methods and materials for treating a neurodegenerative disorder in a mammal by administering a cell penetrating compound, such as 2′-O-methyl oligoribonucleotides having the ability to bind to the active miRNA sequence to the mammal such that the level of miRNA suppression of GRN polypeptide expression in the mammal is reduced. It can be appreciated that miRNA suppression can be reduced by expressing a nucleic acid that comprises a sequence substantially complementary to a pri-miRNA, pre-miRNA, miRNA, or a variant thereof using a viral vector or other appropriate nucleic acid construct. The nucleic acid can be an anti-miRNA. The anti-miRNA can hybridize with a pri-miRNA, pre-miRNA, or miRNA, thereby reducing its gene repression activity. Expression of the target gene can be increased by expressing a nucleic acid that is substantially complementary to a portion of the binding site in the target gene, such that binding of the nucleic acid to the binding site prevents miRNA binding. Having the ability to treat neurodegenerative disorders can help clinicians reduce the considerable morbidity and mortality associated with such disorders, and can also reduce health care expenditures.

In general, one aspect of this document features a method for diagnosing dementia in a mammal suspected of having dementia. The method comprises, or consists essentially of, determining whether or not the mammal contains a mutant T allele of rs5848, wherein the mammal has dementia if the mammal contains the mutant T allele. The mammal can be a human. The dementia can be frontotemporal lobar degeneration. The dementia can be frontotemporal dementia. The method can comprise determining whether or not the mammal is heterozygous for the mutant T allele, wherein the mammal has dementia if the mammal is heterozygous for the mutant T allele. The method can comprise determining whether or not the mammal is homozygous for the mutant T allele, wherein the mammal has dementia if the mammal is homozygous for the mutant T allele.

In another aspect, this document features a method for classifying a mammal as being at risk of developing dementia. The method comprises, or consists essentially of, determining whether or not a mammal contains a mutant T allele of rs5848, wherein that the mammal is at risk of developing dementia if the mammal contains the mutant T allele. The mammal can be a human. The dementia can be frontotemporal lobar degeneration. The dementia can be frontotemporal dementia. The method can comprise determining whether or not the mammal is heterozygous for the mutant T allele, wherein the mammal is at risk of developing dementia if the mammal is heterozygous for the mutant T allele. The method can comprise determining whether or not the mammal is homozygous for the mutant T allele, wherein the mammal is at risk of developing dementia if the mammal is homozygous for the mutant T allele.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 contains an overview of genetic variants included in the GRN association studies. Each of the 13 genetic variants included in the association studies are shown relative to their position on chromosome 17q21.31. SNP rs5848 located in the 3′UTR of GRN and strongly associated with FTLD-U is shown in red, while all other SNPs included in the GRN genomic region are in blue. SNPs from the downstream haplotype block are in green.

FIG. 2 contains in silico analyses of the pairing of miR-659 to the predicted binding site in the 3′UTR of GRN. (A) Base-pairing in the presence of wild-type C-allele at rs5848 or (B) risk T-allele at rs5848. The presence of the risk T-allele is expected to lead to the formation of three additional base-pairs at the 5′end of miR-659 compared to the wild-type C-allele, resulting in a stronger binding and more efficient inhibition of GRN translation.

FIG. 3 contains a correlation of rs5848 genotypes with PGRN expression levels in vivo. (A) Representative immunoblot analyses of cerebellar tissue samples of FTLD-U patients, showing reduced expression of PGRN in homozygous T-allele carriers compared to homozygous C-allele carriers. (B) Quantification of immunoblotted PGRN in cerebellar tissue samples of FTLD-U patients homozygous for the rs5848 C-allele or T-allele (N=7 in each group). PGRN expression was normalized to GAPDH. Data represent mean±SEM. (** indicates p<0.001; two sample t-test). (C) Representative PGRN immunohistochemistry observed in granular cell layer of the cerebellum in FTLD-U patients homozygous for the wild-type C-allele of rs5848 (left panel) or homozygous for the risk T-allele (right panel). Scale=50 μM. (D) Quantification of the PGRN burden in granular cell layer of the cerebellum in FTLD-U patients using immunohistochemistry (N=5 in each group). PGRN immunoreactivity was ˜50% lower in FTLD-U patients homozygous for the risk T-allele compared to homozygous C-allele carriers. (** indicates p<0.001, two sample t-test). (E) Relative PGRN mRNA expression determined by quantitative RT-PCR in cerebellar brain samples of FTLD-U patients homozygous for the rs5848 C-allele or T-allele (N=7 in each group). No significant difference in mRNA expression levels was observed. Data represent mean±SEM.

FIG. 4 contains a correlation of rs5848 genotypes with GRN expression levels in vivo. (A) Representative immunoblot analyses of 6 cerebellar tissue samples of FTLD-U patients with the indicated genotypes, showing reduced expression of GRN in rs5848 TT compared to CC carriers. A loss-of-function GRN mutation carrier (Mut) is included for comparison. (B) Quantification of immunoblotted GRN in cerebellar tissue samples of FTLD-U rs5848 TT or CC carriers (N=7 in each group). GRN expression was normalized to GAPDH. Data represent mean±SEM. (** indicates p<0.001; two sample t-test). (C) Quantification of GRN protein levels by ELISA in the TBS-X fraction of cerebellar tissue samples of FTLD-U rs5848 TT and CC carriers (N=7 in each group). GRN expression was ˜30% lower in FTLD-U rs5848 TT compared to CC carriers. Four loss-of-function GRN mutation carriers are included for comparison. (** indicates p<0.001; two sample t-test). (D) Relative GRN mRNA expression determined by quantitative RT-PCR in cerebellar brain samples of FTLD-U rs5848 TT or CC carriers (N=7 in each group). No significant difference in mRNA expression levels was observed. Data represent mean±SEM.

FIG. 5 contains functional analyses of the GRN regulation by miR-659 in vitro. (A) Representative immunoblot analyses of human M17 cells transfected with miR-659 or control miRNAs (miR-C1 and miR-C2). (B) Quantification of immunoblotted GRN in human M17 cells transfected with miR-659 or control miRNAs. GRN expression was normalized to GAPDH. Data are from four independent experiments and represent mean±SEM. (** indicates p<0.001; two sample t-test). (C) Schematic overview of the pMIR-REPORT vector containing a CMV promoter, firefly luciferase gene, full-length 3′UTR of GRN and SV40 poly A-tail. Constructs with wild-type C-allele and risk T-allele of rs5848 at position 78 in 3′UTR were created as well as a Δ18 construct in which the complete predicted binding site of miR-659 was deleted (position 71-88 in the 3′UTR). (D) Luciferase expression in N2A cells transfected with pMIR-REPORT-rs5848C or pMIR-REPORT-Δ18 and co-transfected with high dose (12 nM) miR-659 or miR-C2. Relative luciferase activity was determined as firefly luciferase activity normalized to Renilla luciferase activity. Each experiment was repeated 6 times. miR-659 significantly decreased the expression of firefly luciferase using the wild-type pMIR-REPORT-rs5848C vector, but not using the pMIR-REPORT-Δ18 in which the miR-659 binding site was deleted. Data represent mean±SEM. (** indicates p<0.001; two sample t-test). (E-F) pMIR-REPORT-rs5848C and pMIR-REPORT-rs5848T vectors were transfected in N2A cells and co-transfected with variable low doses (0.01 pM-100 pM) of miR-659 or miR-C2. Each experiment was repeated 3 times. A significant reduction in the expression of firefly luciferase was observed in the presence of 5 pM and 100 pM of miR-569 from the rs5848T construct (D, ** p<0.02; two-tailed t-test), while no reduction in the expression of firefly luciferase from the rs5848C construct was observed at any of the doses. Data represent mean±S.E.M. (E) pMIR-REPORT-rs5848T vector was transfected in N2A cells and co-transfected with variable doses (0.01 pM-100 pM) of either miR-659 or a random 23-base pair control miRNA. Each experiment was repeated 3 times. A significant reduction in the expression of firefly luciferase from the pMIR-REPORT-rs5848T was observed in the presence of 5 pM and 100 pM of miR-569, while a non-significant reduction was observed at 20 pM (p=0.06). Data represent mean±SEM. (** indicates p<0.02; two sample t-test). (F) pMIR-REPORT-rs5848C was transfected in N2A cells and co-transfected with variable doses (0.01 pM-100 pM) of either miR-659 or a random 23-base pair control miRNA. Each experiment was repeated 3 times. No reduction in the expression of firefly luciferase from the pMIR-REPORT-rs5848C was observed at any of the doses. Data represent mean±SEM.

FIG. 6 contains a pathological characterization of FTLD-U patients stratified by rs5848 genotype. (A) Stratified by rs5848 genotype, the percentage of patients with each FTLD-U pathological subtype is shown. FTLD-U patients homozygous for the risk T-allele show the highest frequency of FTLD-U type 1. (B) Stratified by rs5848 genotype, the percentage of patients with characteristic lentiform NIIs is shown. FTLD-U patients homozygous for the risk T-allele present significantly more frequent with NIIs compared to heterozygous CT and homozygous CC carriers (p=0.04, Fisher exact test).

FIG. 7 contains a pathological characterization of FTLD-U patients stratified by rs5848 genotype. (A) Stratified by rs5848 genotype, the percentage of patients with each FTLD-U pathological subtype is shown. FTLD-U patients homozygous for the risk T-allele show the highest frequency of FTLD-U type 1. (B) Stratified by rs5848 genotype, the percentage of patients with characteristic lentiform NIIs is shown. FTLD-U patients homozygous for the risk T-allele present significantly more frequent with NIIs compared to heterozygous CT and homozygous CC carriers (p=0.02, Fisher exact test).

### DETAILED DESCRIPTION

This description provides methods and materials related to determining whether or not a mammal contains zero, one, or two copies of the mutant ‘T’ allele of rs5848. For example, this description provides methods and materials for determining whether or not a mammal is homozygous or heterozygous for the mutant ‘T’ allele of rs5848. As described herein, a mammal is homozygous or, in some cases heterozygous, for the mutant ‘T’ allele of rs5848 can be identified as having or as being likely to develop dementia.

The mammal can be any type of mammal including, without limitation, a mouse, rat, dog, cat, horse, sheep, goat, cow, pig, monkey, or human. Examples of GRN nucleic acid include, without limitation, the nucleic acid sequence set forth in GenBank® Accession Number M75161 (GI:183612).

The methods and materials provided herein can be used to determine whether or not a GRN nucleic acid of a mammal (e.g., human) contains the mutant ‘T’ allele of rs5848. In some cases, the methods and materials provided herein can be used to determine whether both alleles containing GRN nucleic acid of a mammal contain the mutant ‘T’ allele of rs5848, or whether only a single allele containing GRN nucleic acid of the mammal contains the mutant ‘T’ allele of rs5848. The identification of the mutant ‘T’ allele of rs5848 can be used to diagnose dementia in a mammal, typically when known clinical symptoms of a neurological disorder also are present. The identification of the mutant ‘T’ allele of rs5848 in only one allele can indicate that the mammal is a carrier.

Any appropriate method can be used to detect the mutant ‘T’ allele of rs5848 in GRN nucleic acid. For example, mutations can be detected by sequencing cDNA, untranslated sequences, denaturing high performance liquid chromatography (DHPLC; underfill et al., Genome Res., 7:996-1005 (1997)), allele-specific hybridization (Stoneking et al., Am. J. Hum. Genet., 48:370-382 (1991); and Prince et al., Genome Res., 11(1): 152-162 (2001)), allele-specific restriction digests, mutation specific polymerase chain reactions, single-stranded conformational polymorphism detection (Schafer et al., Nat. Biotechnol., 15:33-39 (1998)), infrared matrix-assisted laser desorption/ionization mass spectrometry (WO 99/57318), and combinations of such methods.

In some cases, genomic DNA can be used to detect the mutant ‘T’ allele of rs5848 in GRN nucleic acid. Genomic DNA typically is extracted from a biological sample such as a peripheral blood sample, but can be extracted from other biological samples, including tissues (e.g., mucosal scrapings of the lining of the mouth or from renal or hepatic tissue). Any appropriate method can be used to extract genomic DNA from a blood or tissue sample, including, for example, phenol extraction. In some cases, genomic DNA can be extracted with kits such as the QIAamp® Tissue Kit (Qiagen, Chatsworth, Calif.), the Wizard® Genomic DNA purification kit (Promega, Madison, Wis.), the Puregene DNA Isolation System (Gentra Systems, Minneapolis, Minn.), or the A.S.A.P.3 Genomic DNA isolation kit (Boehringer Mannheim, Indianapolis, Ind.).

An amplification step can be performed before proceeding with the detection method. For example, the 3′ UTR of a GRN nucleic acid can be amplified and then directly sequenced. Dye primer sequencing can be used to increase the accuracy of detecting heterozygous samples.

As described herein, the presence of the mutant ‘T’ allele of rs5848 in GRN nucleic acid in a mammal (e.g., human) can indicate that that mammal has dementia. In some cases, the presence of the mutant ‘T’ allele of rs5848 in GRN nucleic acid in a human can indicate that that human has dementia, especially when that human is between the ages of 35 and 75, has a family history of dementia, and/or presents symptoms of dementia. Symptoms of dementia can include changes in behavior such as changes that result in impulsive, repetitive, compulsive, or even criminal behavior. For example, changes in dietary habits and personal hygiene can be symptoms of dementia. Symptoms of dementia also can include language dysfunction, which can present as problems in expression of language, such as problems using the correct words, naming objects, or expressing oneself. Difficulties reading and writing can also develop. In some cases, the presence of GRN nucleic acid containing the mutant ‘T’ allele of rs5848 in a mammal, together with positive results of other diagnostic tests, can indicate that the mammal has dementia. For example, the presence of two mutant ‘T’ alleles of rs5848 together with results from a neurological exam, neurophysical testing, cognitive testing, and/or brain imaging can indicate that a mammal has dementia. Other diagnostic tests can include, without limitation, tests for mutations in MAPT and/or apolipoprotein E (APOE) nucleic acid. In some cases, the presence of one or two mutant ‘T’ alleles of rs5848 in a mammal can indicate that the mammal has neuropathy (e.g., ub-ir lentiform neuronal intranuclear inclusions (NII) in the neocortex and striatum, moderate to severe superficial laminar spongiosis in the neocortex, chronic degenerative changes in the neocortex, ub-ir neurites in the neocortex, well-defined ub-ir neuronal cytoplasmic inclusions (NCI) in the neocortex, numerous ub-ir neurites in the striatum, NCI in the hippocampus with a granular appearance, or any combination thereof (Mackenzie et al., Brain, 129(Pt 11):3081-90 (2006)).

In some cases, any mammal containing one or two mutant ‘T’ alleles of rs5848 can be classified as having an elevated risk of developing dementia. For example, a human having one or two mutant ‘T’ alleles of rs5848 can be classified as having an elevated risk of developing dementia when the human is any age (e.g., less than 65, 60, 55, 50, 45, 40, or 35 years old), does or does not appear to have symptoms of dementia, or has or has not had a positive or negative diagnostic test for dementia. In some cases, a human having one or two mutant ‘T’ alleles of rs5848 can be classified as having an elevated risk of developing dementia when the human also has one or more mutations in MAPT or APOE nucleic acid and is less than, for example, 35 years old or does not appear to have symptoms of dementia.

This document also provides methods and materials related to treating mammals (e.g., humans) having or being likely to develop (e.g., having an elevated risk of developing) a neurodegenerative disorder such as dementia. A mammal can be identified as having or being likely to develop a neurodegenerative disorder (e.g., frontotemporal dementia) if it is determined that the mammal one or two mutant ‘T’ alleles of rs5848. A neurodegenerative disorder can be any condition in which neurons are damaged. Examples of neurodegenerative disorders include, without limitation, AD, dementia, frontotemporal dementia (FTD), FTLD, Parkinson's disease, Huntington's disease, stroke, and motor neuron disease.

After identifying a mammal as having or being likely to develop a neurodegenerative disorder, a health-care professional can take one or more actions that can affect the mammal's care. For example, a health-care professional can record information regarding GRN expression levels or the presence of one or two mutant ‘T’ alleles of rs5848 in a mammal's medical record. In some cases, a health-care professional can record a diagnosis of having or being likely to develop a neurodegenerative disorder (e.g., frontotemporal dementia), or otherwise transform the mammal's medical record, to reflect the mammal's medical condition. In some cases, a health-care professional can review and evaluate a mammal's medical record, and can assess multiple treatment strategies for clinical intervention of a mammal's condition.

A health-care professional can initiate or modify treatment for a neurodegenerative disorder (e.g., frontotemporal dementia) after receiving information regarding GRN expression levels or the presence of one or two mutant ‘T’ alleles of rs5848. As described herein, a mammal identified as having or being susceptible to developing a neurodegenerative disorder can be treated by administering a cell penetrating compound, such as 2′-O-methyl oligoribonucleotides having the ability to bind to the active miRNA sequence to the mammal such that the level of miRNA suppression of PGRN polypeptide expression in the mammal is reduced. It can be appreciated that miRNA suppression can be reduced by expressing a nucleic acid that comprises a sequence substantially complementary to a pri-miRNA, pre-miRNA, miRNA, or a variant thereof using a viral vector or other appropriate nucleic acid construct. The nucleic acid can be an anti-miRNA. The anti-miRNA can hybridize with a pri-miRNA, pre-miRNA, or miRNA, thereby reducing its gene repression activity. Expression of the target gene can be increased by expressing a nucleic acid that is substantially complementary to a portion of the binding site in the target gene, such that binding of the nucleic acid to the binding site prevents miRNA binding. In some cases, chemically modified, cholesterol-conjugated single-stranded RNA analogues complementary to miRNAs (e.g., oligonucleotides “antagomirs” (Krützfeldt et al., Nature, 438:685-689 (2005))) can be used to reduce miRNA suppression of GRN polypeptide expression.

The invention will be further described in the following examples, which do not limit the scope of the invention described in the claims.

### EXAMPLES

### Example 1

### A Common Variant in a miRNA Binding Site of Progranulin is a Risk Factor for Ubiquitin-Positive Frontotemporal Lobar Degeneration

Mayo Clinic FTLD case-control series. The Mayo Clinic FTLD patient series in which the initial observation of a deviation from Hardy-Weinberg equilibrium (HWE) for rs5848 was observed is described elsewhere (Gass et al., Hum. Mol. Genet., 15:2988-3001 (2006)). A total of 934 control individuals (mean age at inclusion 64.8±10.9 years) ascertained through MCJ and MCS were employed to determine initial rs5848 control genotype frequencies.

FTLD-U case-control series. The Mayo Clinic Jacksonville (MCJ) brain bank comprises >2500 neurodegenerative brain samples. All 81 patients from the MCJ brain bank with the neuropathological diagnosis of FTLD-U and positive TDP-43 immunostaining were selected. Of these, 19 patients with a GRN loss-of-function mutation, one VCP mutation carrier and one LRRK2 mutation carrier were excluded from the study, resulting in a total of 59 FTLD-U patients for genetic and functional analyses. In this series, the mean age at death was 74.4±9.8 years (range 56-97 years). A selection of 433 control individuals matched for age and gender to the FTLD-U patient population were drawn from two large cohorts of unrelated control individuals collected at MCJ and MCS. The average age at inclusion of the study was 74.5±5.7 years (range 65-87 years).

Genotyping analyses. Genotyping of all additional SNPs selected for association and LD structure analyses, with the exception of rs34424835, was performed using pre-designed and custom TaqMan SNP genotyping assays (Applied Biosystems) and analyzed on an ABI 7900HT Fast Real Time PCR system using the SDSv2.2.2 software (see Table 1). A pre-designed TaqMan SNP genotyping assay was also used to confirm the rs5848 genotyping observed by sequencing the Mayo Clinic FTLD patient series and to determine the genotype frequencies of rs5848 in the FTLD-U and control individuals.

TABLE 1
Information on Tagman genotyping assays

| Position relative | | | | |
|---|---|---|---|---|
| to GRN | dbSNP ID | ABI TaqMan Assay | PCR Primers | Reporter primers (Dye) |
| GRN c. − 4671C > T | rs4792937 | Custom assay | F: GAGCGAGCCAGCTCAGTAG | CTGGGATTACAAATGTGAG |
| | | | R: GCTCCCAAAGCGATTCTCCTA | (VIC) |
| | | | | CTGGGATTACAAGTGTGAG |
| | | | | (FAM) |
| GRN c. − 2977C > T | rs2879096 | C_15835934_10 | N/A | N/A |
| GRN c. − 327C > G | N/A | Custom assay | F: CTGCACAGATCAGACCCACAA | TCAGGAAGACGTGATTT |
| | | | R: CACTGGGCAGGCTTATGAGA | (VIC) |
| | | | | TCAGGAAGACCTGATTT |
| | | | | (FAM) |
| GRN | rs9897526 | C_2548248_10 | N/A | N/A |
| c.264 + 21G > A | | | | |
| GRN c.384T > C | rs25646 | C_14929_10 | N/A | N/A |
| (D128D) | | | | |
| GRN c.835 + 7G > A | N/A | Custom assay | F: ACGGACCTCCTCACTAAGCT | ACAGGTACCAGAGGCA (VIC) |
| | | | R: GCCCCACCCCTGTATCTG | AGGTACCAAAGGCA (FAM) |
| GRN c.*78C > T | rs5848 | C_7452046_20 | N/A | N/A |
| Downstream of | rs708384 | C_2259468_10 | N/A | N/A |
| GRN | | | | |
| Downstream of | rs850737 | C_2548239_10 | N/A | N/A |
| GRN | | | | |
| Downstream of | rs5910 | C_2548233_10 | N/A | N/A |
| GRN | | | | |
| Downstream of | rs5911 | C_3017440_10 | N/A | N/A |
| GRN | | | | |
| Downstream of | rs850730 | C_11880722_20 | N/A | N/A |
| GRN | | | | |

To genotype the rs34424835 deletion polymorphism, marker D17S1860 and the newly developed marker GRN_GT15, each marker was PCR amplified with one fluorescently labeled primer, and analyzed on an automated ABI3100 DNA-analyzer (Table 2). Alleles were scored using the GENOTYPER software (Applied Biosystems).

TABLE 2
Primers for marker genotyping assays

| | UCSC genome browser | |
|---|---|---|
| Marker | position (Mar. 2006) | Primers |
| rs34424835 | chr17:39783076-39783075 | F: TAGTGTCACCCTCAAACCCCAGT-FAM |
| | | R: CCACGGAGTTGTTACCTGTGGAC |
| D17S1860 | chr17:39725782-39726150 | F: GCGCAATCTCAGGTCA-FAM |
| | | R: ACCACCGTGTCTGGCTA |
| GRN_GT15 | chr17:39790625-39790652 | F: TCCCATTTCTCCCTTCTAGTTG-FAM |
| | | R: AAGTTGAGGCTGCAGGGTG |

Single SNP and haplotype association analyses. In the FTLD-U patient-control series, single SNP age- and gender adjusted logistic regression analyses were performed using the StatsDirect statistical software program (available at statsdirect.com on the world wide web). A genotypic model was used to determine the risk associated with carrying one or two rare alleles, using homozygote carriers of the frequent allele as a reference. Haplotype association was analyzed using a score method described elsewhere with adjustments made for age and gender (Schaid et al., Am. J. Hum. Genet. 70:425-34 (2002)). Only haplotypes with an estimated overall frequency of ≧5% were considered in the analyses. The level of significance was defined as p<0.05.

Statistical analyses of rs5848. In the FTLD-U patient-control series, age- and gender-adjusted logistic regression analyses were performed using the StatsDirect statistical software program (see, e.g., internet at “statsdirect.com”). A genotypic model was used to determine the risk associated with carrying the CT or TT genotype of rs5848, using CC genotype carriers as a reference. Chi-Square tests were used to compare the difference in rs5848 genotypic distributions in the clinical FTLD case-control series.

Estimation of haplotype frequencies in GRN genomic region. The expectation maximization algorithm provided by the Arlequin package was applied in order to estimate GRN haplotype frequencies in 35 FTLD patients homozygous for the rs5848 T-allele.

Immunoblot analyses. To determine GRN expression in human brain, 14 cerebellar brain samples of FTLD-U cases (7 homozygous C-allele carriers and 7 homozygous T-allele carriers) were sonicated in PARIS cell disruption buffer (Ambion/Applied Biosystems, Austin, Tex.) supplemented with protease and phosphatase inhibitors, centrifuged, and the protein concentrations determined with a BCA protein assay (Pierce, Rockford, Ill.). 40 μg of protein were resolved by SDS-PAGE using pre-cast 8% Tris-Glycine gels (Invitrogen, Carlsbad, Calif.). Separated proteins were transferred to PVDF membranes and blocked for 1 hour at room temperature with 5% skim milk/TBST. After incubation with anti-human PCDGF (Zymed, South San Francisco, Calif.) primary antibody under blocking conditions, proteins were detected with anti-rabbit HRP conjugated secondary antibody (Southern Biotech, Birmingham, Ala.) and ECL-Plus (Perkin Elmer, Waltham, Mass.). Quantification of immunoreactive bands was performed by densitometry (Image J, Research Services Branch, NIMH, Bethesda, Md.). GRN protein levels were normalized to GAPDH by re-probing blots with anti-GAPDH (1:50,000; Biodesign International, Saco, Me.) and anti-mouse HRP conjugated secondary (1:25,000: Southern Biotech) antibody. To compare GRN protein levels assessed on separate immunoblots, a further normalization was performed using a reference sample included on each blot. In addition, all immunoblots were run in parallel on the same day, to minimize variability in experimental conditions.

To determine GRN expression in human M17 cells transiently transfected with miR-659 or negative control miRNAs (Cye-3 dye labeled miRNA Negative Control #1 (miR-C1) and miRNA Negative Control #2 (miR-C2), Ambion), M17 cells were plated on TC-treated COSTAR® 6-well cell culture plates (Corning, Inc. Corning, N.Y.) at 3.0×10⁵ cells per well in antibiotic-free Opti-mem reduced serum medium supplemented with 10% fetal bovine serum (Invitrogen). Four hours after plating, cells were transfected with Lipofectamine 2000 (Invitrogen) using manufacturer's instructions, with miR-659, miR-C1, or miR-C2, all at 12 nM. Three replicates were performed for each treatment. 48 hours after transfection, cells were harvested, lysed in a mild detergent buffer containing protease and phosphatase inhibitors, sonicated and centrifuged. SDS-PAGE and immunoblot analyses were performed as described above using 6 μg of protein. Immunoblot analysis was performed four times for each treatment group. Statistical analyses were performed using two-tailed t-tests.

GRN ELISA assay. To further quantify GRN expression in human brain, cerebellar brain samples of 25 FTLD-U cases (7 FTLD-U cases with each of the rs5848 genotypes and 4 loss-of-function GRN mutation carriers) were homogenized in 1×TBS (Boston Bioproducts Inc., Worcester, Mass.) supplemented with protease and phosphatase inhibitors and centrifuged for 5 minutes at 16,000 g at 4° C. The supernatant was saved as the TBS fraction and the pellet was resuspended and sonicated in TBS-X (1×TBS with 0.1% Triton X-100). After centrifugation (5 minutes at 16,000 g at 4° C.), the supernatant was saved as the TBS-X fraction. The protein concentration of each fraction was determined using a BCA protein assay (Pierce). For each sample, 100 μg of protein of each fraction was analyzed in duplicate using a GRN ELISA assay (Human Progranulin ELISA Kit, AdipoGen Inc, Seoul, Korea). Recombinant human GRN provided with the ELISA kit was used as a standard.

FTLD-U subtyping. Glass-mounted sections (5 μm thick) of formalin-fixed, paraffin-embedded tissue from multiple brain regions (frontal cortex, temporal cortex, hippocampus, amygdala, basal ganglia and medulla) were immunostained for TDP-43 (rabbit polyclonal antibody; 1:3,000; ProteinTech Group, Inc., Chicago, Ill.) with a DAKO-Autostainer (DAKO-Cytomaton, Carpinteria, Calif.) and 3,3′-diaminobenzidine as the chromogen. Sections were lightly counterstained with hematoxylin. The distribution of neuronal cytoplasmic inclusions (NCIs), dystrophic neurites and neuronal intranuclear inclusions (NIIs) were noted in each section, and a FTLD-U subtype was assigned as described elsewhere (Mackenzie et al., Acta. Neuropathol., 112:539-49 (2006)). Presence or absence of NIIs and motor neuron disease (MND) was also recorded. Type 1 cases exhibited pleomorphic NCIs and short, thin neurites in upper cortical layers, as well as pleomorphic NCIs in the hippocampal dentate fascia, amygdale, and striatum. There was variable fine neuritic pathology in the hippocampal pyramidal layer. Hypoglossal neurons were not affected in sections of the medulla, but NCIs were sometimes found in the inferior olive. Most type 1 cases had lentiform NIIs. Type 2 cases had long thick dystrophic neurites in the cortex that did not show a clear laminar distribution, with sparse cortical NCIs. In contrast to paucity of NCIs in the cortex, round NCIs were prominent in the dentate fascia and striatum, but not in lower levels of the neuraxis. Motor neuron pathology was absent. Type 3 cases had pleomorphic NCIs in the cortex, including neurons with granular cytoplasmic TDP-43 immunoreactive that did not form a discrete inclusion (“pre-inclusions”). The amygdala was often affected, but basal ganglia pathology was minimal. Neurons in the hypoglossal nucleus often had TDP-43 immunoreactivity.

The frequency of pathological FTLD-U subtypes and number of FTLD-U patients with NIIs in each of the rs5848 genotype groups was compared using Fisher exact tests.

GRN immunohistochemistry and image analysis. Transverse sections of the cerebellum, including cortex, white matter, and dentate nucleus, were sampled in 10 FTLD-U cases (5 homozygous C-allele carriers and 5 homozygous T-allele carriers) previously included in the immunoblot analyses. To maintain staining consistency, paraffin-embedded sections (5 μm thick) were immunostained with a DAKO-Autostainer using a primary antibody to human GRN (anti-human progranulin, 1:600, R&D Systems, Inc. Minneapolis, Minn.) and 3,3′-diaminobenzidine as the chromogen. Sections were lightly counterstained with hematoxylin. To obtain a quantitative measure of GRN immunoreactivity by image analysis, immunostained slides were converted into high-resolution digital images using an Aperio slide scanner (Aperio Technologies, Vista, Calif.). Blinded to genotypic information, GRN immunoreactivity was quantified in the granular cell layer of the cerebellum, using a positive pixel count algorithm (Imagescope version 8; Aperio Technologies, Vista, Calif.) and expressed as a percentage of the total area (GRN burden). Statistical comparisons were performed using a two-tailed t-test.

Real-time GRN mRNA expression analyses. Total RNA was isolated from cerebellum of 14 FTLD-U patients (7 homozygous C-allele carriers and 7 homozygous T-allele carriers) using the Trizol Plus RNA Purification System (Invitrogen), and its quality assessed on an Agilent 2100 Bioanalyzer. Only RNA samples with RNA integrity (RIN) values >6 were included in the analyses. RNA samples were normalized to 500 ng/μL and using 3 μg as template, and a reverse transcription reaction was performed using a 2:1 mix of random hexamers and oligo(dT) primers and the SuperScript III system (Invitrogen). For expression analyses, Applied Biosystems assays were used for PGRN (Hs00173570 ml), and for the endogenous controls GAPDH (Hs00266705_g1), YWHAZ (Hs00852925_sH), and HPR1 (Hs99999909_m1). Real-time PCR was performed on an ABI 7900 using the TaqMan® real-time PCR method. Reactions contained 1 μL cDNA amplified with 0.25 μL primer/probe mix and 2.5 μL TaqMan 2× Universal PCR Master Mix. The cycling parameters as recommended by the manufacturer were followed: 50° C. for 2 minutes, 95° C. for 10 minutes, followed by 40 cycles of 95° C. for 15 seconds/60° C. for 1 minute. All samples were run in triplicate and normalized to the geometric mean of the three endogenous controls as described elsewhere (Vandesompele et al., Genome Biol., 3(7):RESEARCH0034.1-RESEARCH0034.11 (2002)). The FAM-fluorescent signal was analyzed using SDSv2.2.2 software, and relative quantities of GRN mRNA were determined using the ΔΔct method.

Generation of human PGRN 3′UTR luciferase constructs. The 3′UTR of PGRN was amplified from genomic DNA from individuals homozygous for the C-allele or T-allele of rs5848 using the following primers:

| | |
|---|---|
| 3UTR-SpeI-F: | |
| AATTACTAGTGGGACAGTACTGAAGACTCTGC | (SEQ ID NO: 1) |
| 3UTR-HindIII-R: | |
| AATTAAGCTTAGTGTACAAACTTTATTGAAACGC | (SEQ ID NO: 2) |

The 5′ end of each primer was designed to include restriction enzyme digest sites (underlined) for subsequent digestion and ligation into the multiple cloning site of the pMIR-REPORT Luciferase vector (Ambion) to create pMIR-REPORT-rs5848C and pMIR-REPORT-rs5848T vectors. PCR reactions were performed in 100 μL using 50 ng genomic template and 20 pmol of each primer. Initial denaturing at 95° C. was followed by 35 cycles of 94° C. for 30 seconds, 60° C.-50° C. touchdown annealing for 30 seconds, and 72° C. for 30 seconds with a final extension at 72° C. for 10 minutes. PCR products were purified using the Qiaquick purification system (Qiagen) both before and after digestion with SpeI and HindIII (New England Biolabs, Ipswich, Mass.).

To create the pMIR-REPORT-Δ18 luciferase vector in which the 18 bp predicted binding site of miR-659 was deleted, site-directed mutagenesis was performed using the QuikChange protocol (Stratagene, La Jolla, Calif.) on a full length PGRN cDNA clone (Invitrogen) using the following primers:

| | |
|---|---|
| Δ18-F: CAGGCCTCCCTAATTCTCCCTGGAC | (SEQ ID NO: 3) |
| Δ18-R: GTCCAGGGAGAATTAGGGAGGCCTG. | (SES ID NO: 4) |

The mutated construct was subsequently used as template for PCR amplification using the 3′UTR-SpeI-F and 3′UTR-HindIII-R primers, digestion and ligation into the pMIR-REPORT Luciferase vector as described herein. Inserts of all constructs were verified by direct sequencing.

Luciferase Assays. N2A neuroblastoma cells were plated on TC-treated Costar® 6-well cell culture plates (Corning) at 1.0×10⁵ cells per well in antibiotic-free Opti-mem reduced serum medium supplemented with 10% fetal bovine serum and 2 mM L-Glutamine (Invitrogen). Four hours after plating, cells were transfected with Lipofectamine 2000 (Invitrogen) using the manufacturer's protocol. For the first experiment, each well was co-transfected with 100 ng of either pMIR-REPORT-rs5848C or 100 ng pMIR-REPORT-Δ18 and 100 ng of pRL-CMV-renilla luciferase (Promega, Madison, Wis.), and treated with either miR-659 or negative control miR-C2 (Ambion) at 12 nM. Six replicates were performed for each treatment. 24 hours after transfection, cells were lysed using 250 μL of Reporter Gene Assay lysis buffer (Roche, Indianapolis, Ind.) and Luciferase Firefly (LA_{F}) and Luciferase Renilla (LA_{R}) activities were measured in triplicate using the dual-luciferase reporter assay (Promega) on a Veritas microplate Luminometer using manufacturer's instructions. To determine the differential regulation of the rs5848 C- or T-allele constructs by miR-659, luciferase experiments were further performed by co-transfection of pMIR-REPORT-rs5848C or pMIR-REPORT-rs5848T with 100 ng of pRL-CMV-renilla luciferase and treatment with increasing but low doses of pre-miR miR-659 and pre-miR miRNA negative control #2 (0.01 pM-100 pM). Three replicates were performed for each treatment. For each well, the relative luciferase activity (RLA) was calculated as RLA=LA_{F}/LA_{R} using the average from three independent measurements. Next, for each quantity of miRNA, the mean RLA was calculated based on all replicates. Statistical analyses using t-tests were performed for each quantity of miRNA by comparing the mean RLA in cells treated with pre-miR miR-659 with the mean RLA in cells treated with pre-miR miRNA negative controls.

Real-time miR-659 expression analyses. Tissue from the cerebellum of four control brains (pathologically normal) was dissected and processed using the mirVana PARIS system (Ambion) to extract RNA enriched for small RNA species. A TaqMan microRNA reverse transcription reaction was performed following the manufacturer's protocol and using the primers specific for miR-659 (RT 1514) and for human control RNU48 (RT1006) (Applied Biosystems). Real Time PCR was performed on the reverse transcription products to confirm the presence of miR-659 in the M17 cells and the control brain; each 5 μL reaction contained 0.33 μL reverse transcription product with 0.25 μL primer/probe mix and 2.5 μL TaqMan 2× Universal PCR Master Mix (Applied Biosystems) and was cycled as recommended by the protocol: 95° C. for 10 minutes, followed by 40 cycles of 95° C. for 15 seconds/60° C. for 1 minute. Samples were run in triplicate and analyzed using SDSv2.2.2 software. This procedure was repeated on enriched RNA extracted from amygdala, occipital lobe, temporal lobe, frontal lobe, hippocampus, caudate and cerebellum tissue from the same control brain to confirm the presence of miR-659 in these various brain regions.

### Results

Association study of rs5848 with FTLD-U. A close inspection of previous sequencing results (Gass et al., Hum. Mol. Genet., 15:2988-3001 (2006)) in the subgroup of non-GRN mutation carriers (N=339) revealed a statistically significant deviation from the expected Hardy-Weinberg equilibrium (HWE) for the common polymorphism rs5848 (p=0.002), which was attributable to an excess of homozygous patients (Table 3). The rs5848 polymorphism was re-genotyped using a pre-designed Taqman genotyping assay. All rs5848 genotypes were confirmed in the FTLD patient series. Subsequent analyses of rs5848 in a large cohort of control individuals revealed a selective increase in the TT genotype frequency in FTLD patients (16%) compared to control individuals (9%) (p_{genotypic}=0.002) (Table 4).

TABLE 3
Hardy-Weinberg calculation of rs5848 in Mayo Clinic FTLD patients

| | Non-GRN FTLD patients (N = 339) | | |
|---|---|---|---|
| rs5848 | Observed | Expected | |
| genotypes | N | N | Overall p-value |
| CC | 160 | 145.4 | 0.002 |
| CT | 124 | 153.2 | |
| TT | 55 | 40.4 | |

TABLE 4
Genotype frequencies of rs5848 in Mayo Clinic FTLD case-control series

| | | | |
|---|---|---|---|
| | Controls | Patients | |
| rs5848 | (N = 934) | (N = 339) | Overall |

| genotypes | N | % | N | % | p-value |
|---|---|---|---|---|---|
| CC | 463 | 49.6 | 160 | 47.2 | 0.002 |
| CT | 384 | 41.1 | 124 | 36.6 | |
| TT | 87 | 9.3 | 55 | 16.2 | |

To further confirm the genetic contribution of rs5848 to the development of FTLD, the analyses was focused on a homogeneous series of patients with a primary neuropathological diagnosis of FTLD-U with confirmed TDP-43-positive neuronal inclusions, derived from the MCJ brain bank and an age- and gender-matched control group. Of the 81 genealogically unrelated FTLD-U patients identified in the MCJ brain bank, 19 (23.5% of the FTLD-U population) carried a pathogenic loss-of-function GRN mutation and were excluded from the study (Table 5). One VCP and one LRRK2 mutation carrier were also excluded, resulting in a total of 59 FTLD-U patients for the genetic studies. Using logistic regression analyses of rs5848, a highly significant association of rs5848 with FTLD-U (p_{corrected}=0.003) was observed, resulting from an increase in the TT genotype frequency of rs5848 in FTLD-U patients (25.4%) compared to control individuals (9.9%) (Table 6). The odds ratio (OR) to develop FTLD-U for carriers homozygous for the minor T-allele of rs5848 compared to homozygous C-allele carriers was 3.18 (p_{adjusted}=0.003; 95% confidence interval (CI): 1.50-6.73) (Table 6). In contrast, individuals heterozygous for rs5848 did not show an increased risk to develop FTLD-U (p_{adjusted}=0.74; OR=1.12; 95% CI: 0.59-2.10). Since MND pathology is rare or absent in GRN loss-of-function mutation carriers, the association excluding patients with MND pathology (N=11) was re-analyzed, which further increased the OR for homozygous T-allele carriers to 3.76 (95% CI: 1.69-8.39; p_{adjusted}=0.001). Comparison of gender, age at death and brain weight of FTLD-U patients by rs5848 genotype groups did not show significant differences (mean age at death was 71.6±7.4 years in CC, 76.0±10.5 years in CT and 75.5±11.3 years in TT carriers).

TABLE 5
GRN loss-of-function mutations identified in MCJ brain bank series

| Patient | Genomic mutation^{a} | Predicted cDNA^{b} | Predicted protein^{c} |
|---|---|---|---|
| F161-1 | g.100068T > C | c.2T > C | p.Met? |
| NA99-175 | g.100092C > A | c.26C > A | p.Ala9Asp |
| NA03-140 | g.100092C > A | c.26C > A | p.Ala9Asp |
| 8536 | g.100129insC | c.63insC | p.Asp22ArgfsX43 |
| F149-1 | g.100205G > A | c.138 + 1G > A (IVS1 + 1G > A) | p.Met? |
| F142-1 | g.100343delA | c.154delA | p.Thr52HisfsX2 |
| 398-5 | g.101168_101171delCAGT | c.388_391delCAGT | p.Gln130SerfsX125 |
| 104-6 | g.101703G > A | c.708 + 1G > A (IVS6 + 1G > A) | p.Val200GlyfsX18 |
| F147-47 | g.102339_102340insTG | c.910_911insTG | p.Trp304LeufsX58 |
| 4713 | g.102340G > A | c.911G > A | p.Trp304X |
| PPA1-1 | g.102516delG | c.998delG | p.Gly333ValfsX28 |
| F129-2 | g.102663delC | c.1145delC | p.Thr382SerfsX30 |
| F153-1 | g.103132_103133insC | c.1395_1396insC | p.Cys466LeufsX46 |
| NA01-249 | g.103306C > T | c.1477C > T | p.Arg493X |
| NA02-297 | g.103306C > T | c.1477C > T | p.Arg493X |

^{a}Numbering relative to the reverse complement of GenBank accession number AC003043.1 and starting at nucleotide 1.
^{b}Numbering according to GenBank accession number NM_002087.2 starting at the translation initiation codon.
^{c}Numbering according to GenPept accession number NP_002078.1.

TABLE 6
Logistic regression analyses of rs5848 in FTLD-U patient-control series

| | Controls | | Patients | | | | |
|---|---|---|---|---|---|---|---|
| rs5848 | (N = 433) | | (N = 59) | | | | |
| genotypes | N | % | N | % | P_{adjusted} | OR | 95% CI |
| CC | 199 | 46.0 | 21 | 35.6 | — | — | — |
| CT | 191 | 44.1 | 23 | 39.0 | 0.74 | 1.12 | 0.59-2.10 |
| TT | 43 | 9.9 | 15 | 25.4 | 0.003 | 3.18 | 1.50-6.73 |

In a previous study of 60 FTLD-U patients, a highly significant association of rs5848 with FTLD-U (p_{corrected}=0.001) was also observed, resulting from an increase in the TT genotype frequency of rs5848 in FTLD-U patients (25.0%) compared to control individuals (9.6%) (Table 7). The odds ratio (OR) to develop FTLD-U for carriers homozygous for the minor T-allele of rs5848 compared to homozygous C-allele carriers was 3.35 (p_{corrected}=0.001; 95% confidence interval (CI): 1.62-6.95). In contrast, individuals heterozygous for rs5848 did not exhibit an increased risk to develop FTLD-U (p_{corrected}=0.45; OR=1.26; 95% CI: 0.68-2.33). The association excluding patients with MND pathology (N=12) was re-analyzed, which further increased the OR for homozygous T-allele carriers to 3.94 (95% CI: 1.79-8.65; p_{corrected}=0.0006). Comparison of gender, age at death, and brain weight of these FTLD-U patients by rs5848 genotype groups did not reveal significant differences (mean age at death was 72.5±8.2 years in CC, 75.8±10.2 years in CT and 75.5±10.9 years in TT carriers).

TABLE 7
Logistic regression analyses of rs5848 in 60 FTLD-U patient-control
series

| | Controls | | Patients | | | | |
|---|---|---|---|---|---|---|---|
| rs5848 | (N = 572) | | (N = 60) | | | | |
| genotypes | N | % | N | % | P_{corrected} | OR | 95% CI |
| CC | 276 | 48.3 | 21 | 35.0 | — | — | — |
| CT | 241 | 42.1 | 24 | 40.0 | 0.45 | 1.26 | 0.68-2.33 |
| TT | 55 | 9.6 | 15 | 25.0 | 0.001 | 3.35 | 1.62-6.95 |

Association study of rs5848 with clinical FTLD. Genotyping of rs5848 in two clinical FTLD case-control series with unknown pathology did not reveal significant association (combined data in Table 8; data on individual series in Tables 9 and 10). Comparison of the TT genotype frequency among distinct FTLD clinical subtypes, including behavioral variant FTD (bvFTD), progressive non-fluent aphasia (PNFA), semantic dementia (SD), and corticobasal syndrome (CBS), revealed the highest TT genotype frequency (14.3%) in the subgroup of patients with PNFA.

TABLE 8
Genotype frequencies of rs5848 in combined clinical FTLD series

| | | rs5848 | | | | | | |
|---|---|---|---|---|---|---|---|---|
| Popu- | | CC | | CT | | TT | | |
| lation | N_{individuals} | N | % | N | % | N | % | p_{genotypic} |
| Con- | 604 | 308 | 51.0 | 250 | 41.4 | 46 | 7.6 | — |
| trols | | | | | | | | |
| All | 313 | 139 | 44.4 | 147 | 47.0 | 27 | 8.6 | 0.17 |
| patients | | | | | | | | |
| FTD | 140 | 66 | 47.1 | 64 | 45.7 | 10 | 7.1 | 0.64 |
| PNFA | 49 | 21 | 42.9 | 21 | 42.9 | 7 | 14.3 | 0.21 |
| SD | 77 | 34 | 44.2 | 37 | 48.1 | 6 | 7.8 | 0.51 |
| CBS | 47 | 18 | 38.3 | 25 | 53.2 | 4 | 8.5 | 0.23 |

TABLE 9
Genotype frequencies of rs5848 in MCJ case-control series

| | | rs5848 | | | | | | |
|---|---|---|---|---|---|---|---|---|
| Popu- | | CC | | CT | | TT | | |
| lation | N_{individuals} | N | % | N | % | N | % | p_{genotypic} |
| Con- | 268 | 135 | 50.4 | 116 | 43.3 | 17 | 6.3 | |
| trols | | | | | | | | |
| All | 169 | 71 | 42.0 | 84 | 49.7 | 14 | 8.3 | 0.22 |
| patients | | | | | | | | |
| FTD | 61 | 26 | 42.6 | 30 | 49.2 | 5 | 8.2 | 0.53 |
| PNFA | 25 | 10 | 40.0 | 12 | 48.0 | 3 | 12.0 | 0.43 |
| SD | 63 | 30 | 47.6 | 30 | 47.6 | 3 | 4.8 | 0.77 |
| CBS | 20 | 5 | 25.0 | 12 | 60.0 | 3 | 15.0 | 0.06 |

TABLE 10
Genotype frequencies of rs5848 in MCR case-control series

| | | rs5848 | | | | | | |
|---|---|---|---|---|---|---|---|---|
| Popu- | | CC | | CT | | TT | | |
| lation | N_{individuals} | N | % | N | % | N | % | p_{genotypic} |
| Con- | 336 | 173 | 51.5 | 134 | 39.9 | 29 | 8.6 | |
| trols | | | | | | | | |
| All | 144 | 68 | 47.2 | 63 | 43.8 | 13 | 9.0 | 0.68 |
| patients | | | | | | | | |
| FTD | 79 | 40 | 50.6 | 34 | 43.0 | 5 | 6.3 | 0.74 |
| PNFA | 24 | 11 | 45.8 | 9 | 37.5 | 4 | 16.7 | 0.41 |
| SD | 14 | 4 | 28.6 | 7 | 50.0 | 3 | 21.4 | 0.12 |
| CBS | 27 | 13 | 48.1 | 13 | 48.1 | 1 | 3.7 | 0.54 |

### Detailed Genetic Analyses in GRN Genomic Region

To study whether rs5848 is the likely functional variant underlying the association with FTLD-U or whether another genetic variant in linkage disequilibrium (LD) with rs5848 could be responsible for the observed association, a detailed genetic analyses of the GRN genomic region was performed. The LD structure of GRN was determined by sequencing 10.2 kb of GRN (UCSC genome browser, chr17:39775969-39785997), including 2 kb upstream of the non-coding exon 0 and the complete 3′UTR, in 24 US individuals. 27 genetic variants were identified, of which 20 were observed more than once in the 48 chromosomes and were considered informative to determine the GRN LD haplotype structure. The first 14 SNPs were part of a single 5 kb haplotype block including the GRN promoter, 2 kb of regulatory sequences, the non-coding exon 0 and intron 0 (defined as GRN promoter haplotype block). In contrast, little LD was observed in the GRN coding region, with the exception of two variants located in close proximity of GRN exon 4 (rs34424835 and rs850713). Importantly, none of the variants was in LD with rs5848 (D′<0.68; r²<0.37). In fact, using the genotype information available in the HapMap project, it was determined that rs5848 is located at the start of another more downstream haplotype block of 24 kb outside of GRN.

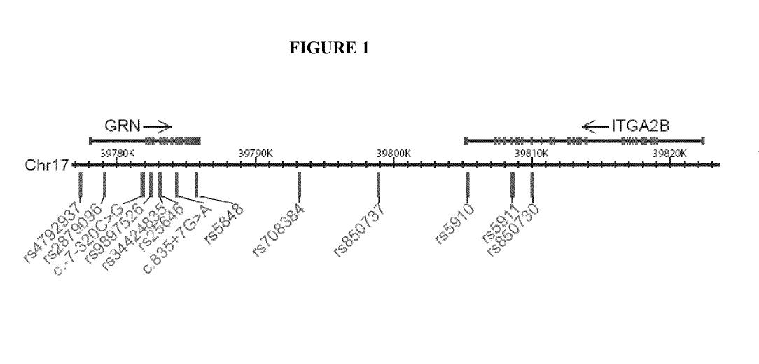

A panel of 13 additional SNPs was selected for single SNP and haplotype association analyses in the FTLD-U patient-control series: 8 tagging SNPs identified in the genomic sequencing analyses that together capture 94% of the genetic diversity in the GRN region and 5 SNPs in considerable LD with rs5848 selected from the downstream haplotype block based on the HapMap data (FIG. 1). Single SNP logistic regression analyses did not identify variants that were more strongly associated with FTLD-U than rs5848, although significant association with 4 of the 5 SNPs from the downstream haplotype block was observed (p_{adjusted}=0.04) (Table 11). Haplotype association analyses adjusted for age and gender did not reveal significant association in the GRN promoter haplotype block, however borderline significance was obtained with Hap C (TCCGinsTGT), carrying the rs5848 risk T-allele, in the complete GRN genomic region (p_{adjusted}=0.04) (Table 12). However, when rs5848 was excluded from the analyses significant association was no longer observed (p_{adjusted}=0.11). Similarly, haplotype analyses in the downstream haplotype block showed a significant increase in Hap B (TACACC) from 30.3% in control individuals to 42.3% in FTLD-U patients (p_{adjusted}=0.02), which was no longer observed when rs5848 was excluded. Sequencing and genotyping analyses further revealed multiple GRN genetic backgrounds for the risk T-allele of rs5848, further favoring rs5848 as the potential functional variant underlying the observed association. Together these results strongly support the hypothesis that rs5848 is the functional variant in GRN associated with FTLD-U, rather than another variant in LD with rs5848 or a haplotype tagged by the T-allele of rs5848.

GRN haplotype diversity in homozygous rs5848 T-allele carriers. To provide additional evidence in favor of rs5848 being the functional variant, it was determined whether the risk T-allele in our patient population is found on different GRN haplotypic backgrounds. 8 genetic variants covering the complete 8 kb GRN genomic region were genotyped in a cohort of 35 FTLD patients from the Mayo Clinic FTLD series that were homozygous for the T-allele at rs5848. Within this population, haplotype estimations using Arlequin revealed 11 different haplotypes, including 8 haplotypes with frequencies >5% (Table 13). Based on the individual genotype data in the patient cohort, only 2 out of 35 FTLD patients (5.7%) were homozygous for the same haplotype (Hap B), while three FTLD patients were homozygous for other GRN haplotypes (one Hap C, one Hap H and one Hap I). When 2 simple tandem repeat (STR) markers flanking GRN (D 17S1860 and GRN_GT15) were included, spanning a total region of 65 kb, haplotype estimation revealed 40 possible haplotypes and none of the 35 patients were homozygous. These data are in agreement with the lack of LD in the GRN genomic region and support the hypothesis that the rs5848 T-allele is found on different GRN haplotypic backgrounds.

TABLE 11
Single SNP association analyses

| | | Controls | | Patients | | | | |
|---|---|---|---|---|---|---|---|---|
| | | (N = 433) | | (N = 59) | | | | |
| SNP | Genotypes | N | % | N | % | P_{adjusted} | OR | 95% CI |
| rs4792937 | TT | 145 | 33.5 | 22 | 37.3 | — | — | — |
| | CT | 210 | 48.5 | 24 | 40.7 | 0.46 | 0.79 | 0.42-1.48 |
| | CC | 78 | 18.0 | 13 | 22.0 | 0.55 | 1.26 | 0.59-2.70 |
| rs2879096 | CC | 244 | 56.4 | 34 | 57.6 | — | — | — |
| | CT | 150 | 34.6 | 18 | 30.5 | 0.72 | 0.90 | 0.48-1.66 |
| | TT | 39 | 9.0 | 7 | 11.9 | 0.38 | 1.50 | 0.61-3.68 |
| c.-7-320C > G | CC | 380 | 87.8 | 53 | 89.8 | — | — | — |
| | CG | 48 | 11.1 | 6 | 10.2 | 0.92 | 0.95 | 0.39-2.35 |
| | GG | 5 | 1.2 | 0 | 0 | n/a | n/a | n/a |
| rs9897526 | GG | 337 | 77.8 | 47 | 79.7 | — | — | — |
| | AG | 90 | 20.8 | 11 | 18.6 | 0.60 | 0.83 | 0.41-1.68 |
| | AA | 6 | 1.4 | 1 | 1.7 | 0.92 | 1.12 | 0.13-9.75 |
| rs34424835 | wt/wt | 245 | 56.6 | 28 | 47.5 | — | — | — |
| | wt/ins | 158 | 36.5 | 25 | 42.4 | 0.29 | 1.37 | 0.77-2.46 |
| | ins/ins | 30 | 6.9 | 6 | 10.2 | 0.25 | 1.77 | 0.67-4.64 |
| rs25646 | TT | 408 | 94.2 | 55 | 93.2 | — | — | — |
| | CT | 25 | 5.8 | 4 | 6.8 | 0.71 | 1.23 | 0.41-3.71 |
| | CC | 0 | 0 | 0 | 0 | n/a | n/a | n/a |
| c.835 + 7G > A | GG | 376 | 86.8 | 51 | 86.4 | — | — | — |
| | AG | 54 | 12.5 | 8 | 13.6 | 0.69 | 1.18 | 0.53-2.64 |
| | AA | 3 | 0.7 | 0 | 0 | n/a | n/a | n/a |
| rs5848 | CC | 199 | 46.0 | 21 | 35.6 | — | — | — |
| | CT | 191 | 44.1 | 23 | 39.0 | 0.74 | 1.12 | 0.59-2.10 |
| | TT | 43 | 9.9 | 15 | 25.4 | 0.003 | 3.18 | 1.50-6.73 |
| rs708384 | CC | 157 | 36.3 | 17 | 28.8 | — | — | — |
| | AC | 210 | 48.5 | 25 | 42.4 | 0.75 | 1.11 | 0.59-2.14 |
| | AA | 66 | 15.2 | 17 | 28.8 | 0.04 | 2.20 | 1.04-4.63 |
| rs850737 | TT | 148 | 34.2 | 18 | 30.5 | — | — | — |
| | CT | 206 | 47.6 | 22 | 37.3 | 0.72 | 0.89 | 0.46-1.72 |
| | CC | 79 | 18.2 | 19 | 32.2 | 0.08 | 1.90 | 0.94-3.86 |
| rs5910 | GG | 151 | 34.9 | 18 | 30.5 | — | — | — |
| | AG | 210 | 48.5 | 22 | 37.3 | 0.73 | 0.89 | 0.46-1.73 |
| | AA | 72 | 16.6 | 19 | 32.2 | 0.04 | 2.13 | 1.05-4.34 |
| rs5911 | AA | 152 | 35.1 | 18 | 30.5 | — | — | — |
| | AC | 209 | 48.3 | 22 | 37.3 | 0.75 | 0.90 | 0.46-1.75 |
| | CC | 72 | 16.6 | 19 | 32.2 | 0.04 | 2.14 | 1.05-4.36 |
| rs850730 | GG | 152 | 35.1 | 18 | 30.5 | — | — | — |
| | CG | 209 | 48.3 | 22 | 37.3 | 0.75 | 0.90 | 0.46-1.75 |
| | CC | 72 | 16.6 | 19 | 32.2 | 0.04 | 2.14 | 1.05-4.36 |

TABLE 12

Haplotype analyses in GRN genomic
region and downstream haplotype block

| | | | |
|---|---|---|---|
| | Control | | |
| | individuals | Patients | |
| Haplotypes^{a} | N = 433 (%) | N = 59 (%) | P_{adjusted}^{b} |

GRN promoter haplotype block:
rs4792937-rs2879096-c. − 7-320C > G

| | | | |
|---|---|---|---|
| | | | 0.81^{c} |
| Hap A TCC | 56.8 | 56.6 | 0.70 |
| Hap B CTC | 26.3 | 26.1 | 0.83 |
| Hap C CCC | 10.1 | 12.4 | 0.54 |
| Hap D CCG | 5.8 | 3.1 | 0.44 |

Complete GRN genomic region:
rs4792937-rs2879096-c.7.320C > G-rs9897526-
rs3442485-rs25646-c.835 + 7G > A-rs5848

| | | | |
|---|---|---|---|
| | | | 0.27^{c} |
| Hap A TCCGwtTGC | 26.3 | 27.0 | 0.21 |
| Hap B CTCGwtTGC | 16.2 | 16.1 | 0.74 |
| Hap C TCCGinsTGT | 7.1 | 13.5 | 0.04 |
| Hap D TCCGwtTGT | 6.7 | 7.3 | 0.24 |
| Hap E TCCGwtTAC | 6.4 | 2.6 | 0.66 |

Complete GRN genomic region excluding rs5848:
rs4792937-rs2879096-c.7.320C > G-rs9897526-
rs3442485-rs25646-c.835 + 7G > A

| | | | |
|---|---|---|---|
| | | | 0.51^{c} |
| Hap A TCCGwtTG | 33.0 | 32.2 | 0.66 |
| Hap B CTCGwtTG | 18.1 | 20.4 | 0.72 |
| Hap C TCCGinsTG | 9.6 | 15.6 | 0.11 |
| Hap D TCCGwtTA | 6.4 | 2.6 | 0.57 |

GRN downstream block:
rs5848-rs708384-rs850737-rs5910-rs5911-rs850730

| | | | |
|---|---|---|---|
| | | | 0.11^{c} |
| Hap A CCTGAG | 57.6 | 47.4 | 0.06 |
| Hap B TACACC | 30.3 | 42.3 | 0.02 |
| Hap C CACACC | 8.9 | 6.8 | 0.53 |

GRN downstream block excluding rs5848:
rs708384-rs850737-rs5910-rs5911-rs850730

| | | | |
|---|---|---|---|
| | | | 0.17^{c} |
| Hap A CTGAG | 57.8 | 48.3 | 0.07 |
| Hap B ACACC | 39.2 | 49.1 | 0.06 |

^{a}Haplotypes with an average frequency <5% were excluded from the analysis. The risk T-allele of rs5848 is shown in bold.
^{b}Simulated p-values corrected for age (age at death in patients, inclusion age for control individuals) and gender.
^{c}Global p-values.

TABLE 13

Estimated frequencies of 8 kb GRN
haplotypes containing the rs5848 T-allele

| | | |
|---|---|---|
| ID | Haplotype^{a} | Frequency (%) |

| | | |
|---|---|---|
| Hap A | T C G G wt T A G T | 26.7 |
| Hap B | T C G G ins T G G T | 18.4 |
| Hap C | C C G G wt T A G T | 11.4 |
| Hap D | C T G G wt T A G T | 10.0 |
| Hap E | C T G G ins T G G T | 7.9 |
| Hap F | C T G A wt T A G T | 7.7 |
| Hap G | T C G A ins T G G T | 6.4 |
| Hap H | C T G A ins T G G T | 5.9 |
| Hap I | T C G G ins T A G T | 2.9 |
| Hap J | C C G A wt C A G T | 1.4 |
| Hap K | C C C G wt T A G T | 1.4 |

^{a}Haplotypes are composed of rs4792937, rs2879096, c.7 − 320C > G, rs9897526, rs34424835, rs25646, rs850713, c.835 + 7G > A, and rs5848 (cDNA numbering relative to NM_002087.2 starting at the translation initiation codon). The risk T-allele of rs5848 is shown in bold.

rs5848 is located in a predicted miRNA binding site of GRN. The rs5848 single base change (c.*78C>T) is located 78 nucleotides downstream of the translation termination codon in the 3′UTR of the GRN transcript in a predicted binding site for the human specific miRNA miR-659 (Table 14). In Table 14, the 3′UTR position starts from first base after TGA stop codon. Target sites were predicted based on version 5 of the miRBASE registry (available at microrna.sanger.ac.uk on the World Wide Web). A total of 70 different miRNAs were predicted to bind to the 304 bp 3′UTR of GRN, but only miR-659 is predicted to be affected by rs5848, located 78 nucleotides downstream of the ATG stop codon. miRNAs are small non-coding RNAs that bind via imperfect base-pairing with target mRNAs to posttranscriptionally modulate their expression (Bartel, Cell, 116:281-97 (2004)). The following was performed to determine if rs5848 increases the risk for FTLD-U by altering the miRNA regulation of the GRN transcript. Previous studies reported a single nucleotide change in the sequence of the target site can be sufficient to affect miRNA regulation (Abelson et al., Science, 310:317-20 (2005) and Clop et al., Nat. Genet., 38:813-8 (2006)).

TABLE 14
Predicted miRNA target sites in GRN 3′UTR

3′UTR
Position

| miRNA | Start | End |
|---|---|---|
| hsa-miR-924 | 1 | 20 |
| hsa-miR-101 | 1 | 14 |
| hsa-miR-604 | 7 | 27 |
| hsa-miR-296-3p | 10 | 29 |
| hsa-miR-516a-5p | 12 | 34 |
| hsa-miR-526b | 12 | 34 |
| hsa-miR-525-5p | 14 | 34 |
| hsa-miR-23a* | 15 | 37 |
| hsa-miR-23b* | 15 | 37 |
| hsa-miR-615-5p | 16 | 38 |
| hsa-miR-193b* | 17 | 38 |
| rno-miR-336 | 30 | 50 |
| hsa-miR-506 | 35 | 54 |
| hsa-miR-342-5p | 58 | 78 |
| hsa-miR-659 | 68 | 89 |
| hsa-miR-198 | 82 | 102 |
| hsa-miR-615-5p | 84 | 105 |
| hsa-miR-939 | 96 | 119 |
| hsa-miR-920 | 99 | 118 |
| hsa-miR-617 | 113 | 135 |
| hsa-miR-136 | 116 | 138 |
| hsa-miR-296-5p | 121 | 141 |
| hsa-miR-151-3p | 128 | 148 |
| hsa-miR-187* | 157 | 178 |
| hsa-miR-96 | 158 | 180 |
| hsa-miR-18b* | 159 | 178 |
| hsa-miR-744* | 161 | 182 |
| hsa-miR-644 | 170 | 189 |
| hsa-miR-142-3p | 170 | 192 |
| hsa-miR-542-5p | 183 | 207 |
| hsa-miR-483-5p | 195 | 215 |
| hsa-miR-674 | 199 | 220 |
| hsa-miR-181a-2* | 200 | 221 |
| hsa-miR-220c | 207 | 229 |
| hsa-miR-140-3p | 211 | 231 |
| hsa-miR-588 | 215 | 233 |
| hsa-miR-193a-3p | 215 | 235 |
| hsa-miR-631 | 216 | 237 |
| hsa-miR-502-3p | 218 | 240 |
| hsa-miR-500* | 218 | 239 |
| has-miR-501-3p | 218 | 240 |
| hsa-miR-519e | 218 | 239 |
| hsa-miR-518c | 219 | 241 |
| hsa-miR-339-3p | 219 | 240 |
| hsa-miR-523 | 219 | 241 |
| hsa-miR-518e | 220 | 240 |
| hsa-miR-519d | 220 | 241 |
| hsa-miR-518a-3p | 220 | 241 |
| hsa-miR-525-3p | 220 | 241 |
| hsa-miR-518f | 221 | 241 |
| hsa-miR-330-3p | 221 | 242 |
| hsa-miR-518d-3p | 221 | 241 |
| hsa-miR-518b | 221 | 241 |
| hsa-miR-524-3p | 221 | 241 |
| hsa-miR-135a* | 228 | 248 |
| hsa-miR-299-3p | 232 | 254 |
| hsa-miR-888* | 243 | 264 |
| hsa-miR-506 | 243 | 263 |
| hsa-miR-30e | 245 | 264 |
| hsa-miR-485-3p | 248 | 270 |
| hsa-miR-377 | 250 | 270 |
| hsa-miR-603 | 250 | 270 |
| hsa-miR-342-3p | 250 | 271 |
| hsa-miR-933 | 253 | 274 |
| hsa-miR-560 | 255 | 275 |
| hsa-miR-574-3p | 261 | 282 |
| hsa-miR-523 | 261 | 283 |
| hsa-miR-487b | 262 | 283 |
| hsa-miR-139-3p | 264 | 285 |
| hsa-miR-19a | 276 | 299 |
| hsa-miR-19b | 276 | 299 |
| hsa-miR-568 | 280 | 300 |
| hsa-let-7g* | 280 | 300 |
| hsa-miR-648 | 284 | 302 |

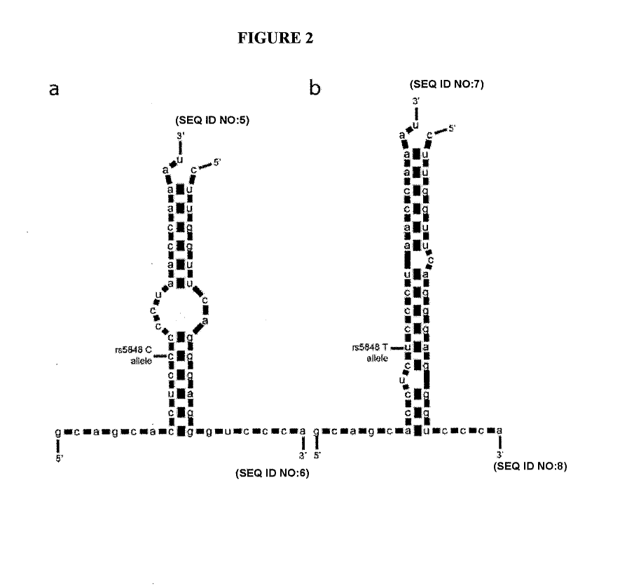

By means of in silico analyses using the RNA folding and two-state hybridization servers (http site at “frontend.bioinfo.rpi.edu/applications/mfold/”), it was predicted that miR-659 binds to the GRN 3′UTR through a perfect complementarity of the ‘seed’ region at position 2-7 of the miRNA and an additional 3′match of an adenosine anchor at position 1 (Zuker, Nucleic Acids Res., 31:3406-15 (2003)). However, depending on the presence of the C-allele or the T-allele at rs5848, the positioning of miR-659 with respect to the miRNA binding site in GRN was expected to shift, resulting in the formation of three additional base-pairs at the 5′end of the miRNA when the risk T-allele of rs5848 was present (FIG. 2). The stronger binding of miR-659 to the GRN mRNA containing the T-allele was expected to result in a more efficient inhibition of GRN translation leading to reduced GRN expression levels.

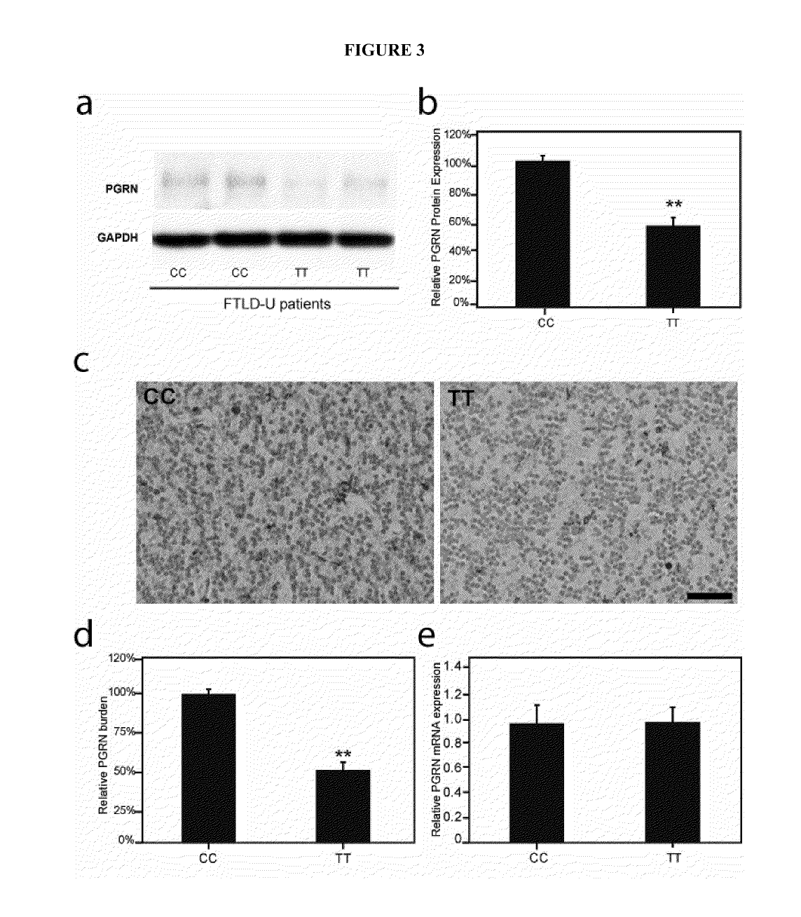

rs5848 affects PGRN polypeptide levels but not mRNA levels in FTLD-U patients. PGRN immunoblot analyses was performed using brain extracts derived from cerebellum of FTLD-U patients homozygous for the C- or T-allele to determine the effect of rs5848 on the expression of PGRN. A significant 46% decrease in PGRN polypeptide levels was observed in homozygous carriers of the T-allele compared to homozygous C-allele carriers (p<0.001) (FIGS. 3A-B). These results were supported by immunohistochemical analysis, which also revealed a significant reduction (˜50%) in PGRN burden in the granular cell layer of the cerebellum in patients homozygous for the T-allele of rs5848 compared to homozygous C-allele carriers (p<0.001) (FIG. 3C-D).

In contrast, PGRN mRNA expression levels as determined by real-time RT-PCR were not significantly different between patients homozygous for the C- or T-allele of rs5848 (FIG. 3E).

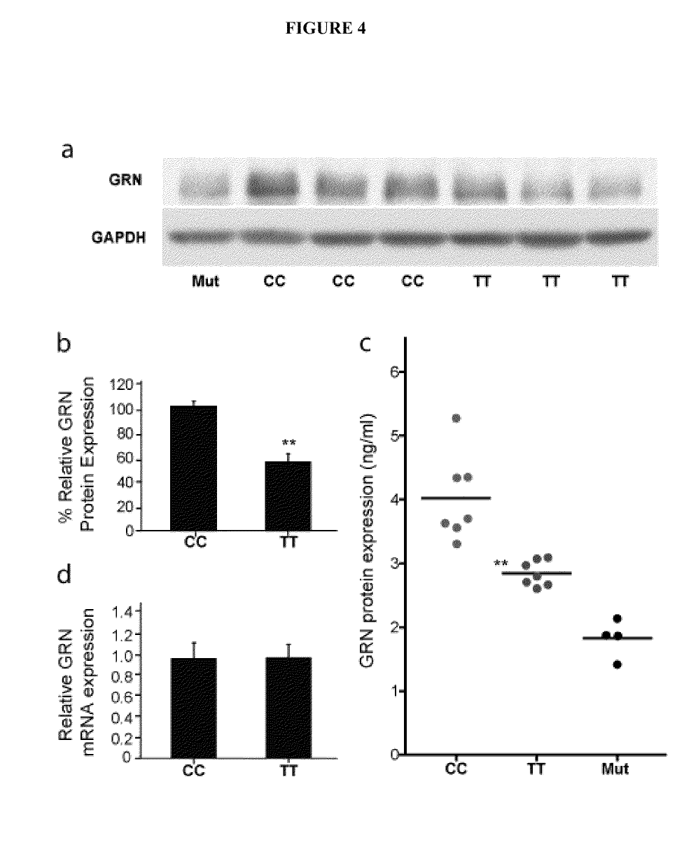

Using Western blot analyses, a significant decrease in GRN protein levels was observed in TT carriers compared to CC carriers (p<0.001). (FIGS. 4A-B). These data were further supported by immunohistochemical analysis, which showed a significant reduction in GRN burden in the granular cell layer of the cerebellum in TT compared to CC patients (p<0.001). (FIG. 4C-D). To further quantify the reduction in GRN, brain homogenates from the cerebellum of FTLD-U rs5848 CC, CT and TT carriers were re-extracted, and GRN expression was determined using an enzyme-linked immunoassay (ELISA). A significant 30% decrease in GRN levels was observed in rs5848 TT carriers compared to CC carriers in the TBS-X fraction (p<0.001) (FIG. 4C). Intermediate levels of GRN protein were observed in FTLD-U patients heterozygous for rs5848 supporting a dose-dependent decrease of GRN with each T-allele.

As expected from a translational repression by miR-659, real-time mRNA expression analyses did not show a significant difference in GRN mRNA levels between rs5848 CC and TT carriers (FIG. 4D).

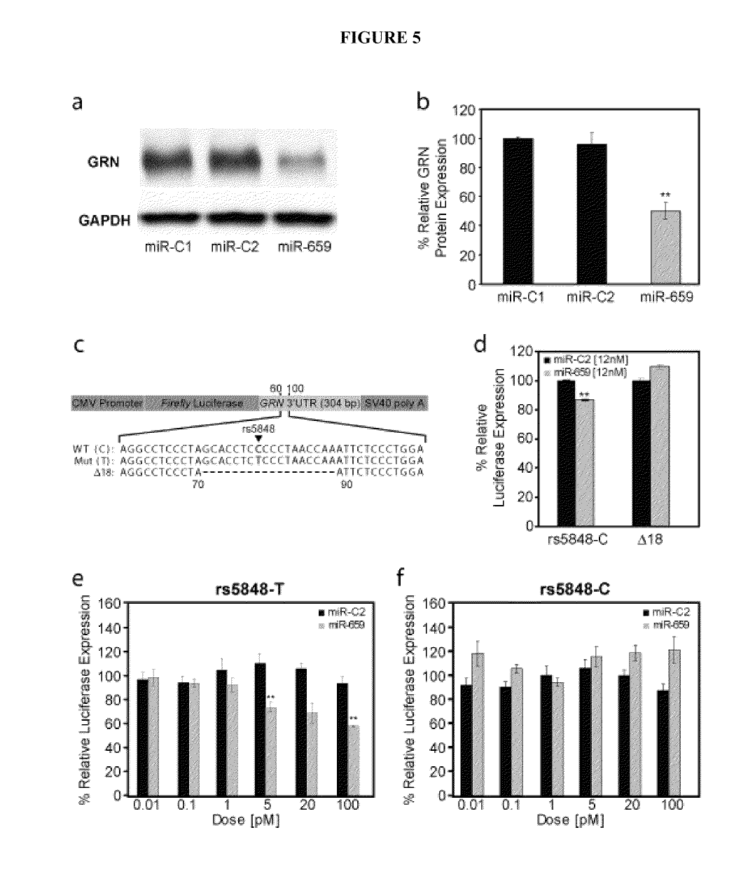

miR-659 inhibits PGRN translation at rs5848. To provide evidence that miR-659 binds to GRN and can regulate the expression of GRN levels in vitro, human M17 cells were transiently transfected with 12 nM of pre-miR miR-659 to mimic the expression of endogenous miR-659, or 12 nM negative control miRNAs, miR-C1 and miR-C2 (Ambion). 48 hours post transfection, cells were harvested, and the endogenous human PGRN polypeptide levels produced by the M17 cells were measured by immunoblot analyses. A highly significant decrease in the expression of endogenous GRN was observed in M17 cells treated with miR-659 (p<<0.001) (FIGS. 5A-B).

To further study the regulation of GRN expression by miR-659, the full-length 304 bp GRN 3′UTR sequence containing the wild-type C-allele was inserted at position 78 downstream of the luciferase reporter gene in the pMIR-REPORT miRNA expression reporter vector system and transiently transfected the construct into mouse N2A neuroblastoma cells (FIG. 5C). Co-transfection of a high dose (12 nM) of miR-659 or negative control miR-C2 in these cells resulted in significantly reduced expression of luciferase (representing GRN protein) in the presence of miR-659 further confirming the functional potential of the mRNA-miRNA duplex (FIG. 4D). A luciferase construct was constructed with the GRN 3′UTR sequence in which the complete 18 bp predicted binding site of miR-659 was deleted (position 71-88 downstream of the termination codon; vectorΔ18) (FIG. 4C). Addition of 12 nM miR-659 to N2A cells transfected with vectorΔ18 failed to repress the luciferase activity (FIG. 5D), supporting the hypothesis that miR-659 binds to the predicted binding-site in the 3′UTR of PGRN, overlapping with rs5848 at position 78.

To further study the differential regulation of PGRN polypeptide expression resulting from the presence of the wild-type ‘C’ or risk ‘T’ allele at rs5848, a luciferase construct containing the PGRN 3′UTR including the risk T-allele at position 78 was created (FIG. 5C). Co-transfection of the luciferase construct containing either the C-allele or the T-allele with variable low doses (0.01 pM-100 pM) of miR-659 or negative control miR-C2 showed a dose-dependent reduction of luciferase activity derived from constructs containing the T-allele, reaching statistical significance at doses of 5 and 100 pM (p<0.02) (FIG. 5E). A strong translational repression of the T-allele construct was also observed at 20 pM, however this result was non-significant (p=0.06). Translational repression of luciferase activity from the wild-type rs5848-C construct was not observed for cells treated with miR-659 at any of these low doses (0.01 pM-100 pM) (FIG. 5F).

miR-659 is expressed in human brain. To confirm the expression of miR-659 in human cells and in brain, a Taqman expression assay specific for miR-659 was used. Positive expression of mature miR-659 was observed in both M17 cells and cerebellum. Additional analyses using RNA extracted from seven different brain regions of a control brain (amygdala, occipital lobe, temporal lobe, frontal lobe, hippocampus, caudate, and cerebellum) showed miR-659 expression in all analyzed regions, including frontal and temporal neocortex, which is most affected in FTLD-U.

Correlation of rs5848 genotypes with FTLD-U pathological subtype. To determine whether the neuropathology of rs5848 TT carriers resembles the pathology of GRN loss-of-function mutation carriers, the FTLD-U pathological subtype was determined as proposed by the classification scheme described elsewhere (Mackenzie et al., Acta. Neuropathol., 112:539-49 (2006)) for all FTLD-U cases included in the genetic analyses of rs5848 with paraffin-embedded tissue blocks available for additional studies (N=57). In addition, the absence or presence of lentiform NIIs was determined.

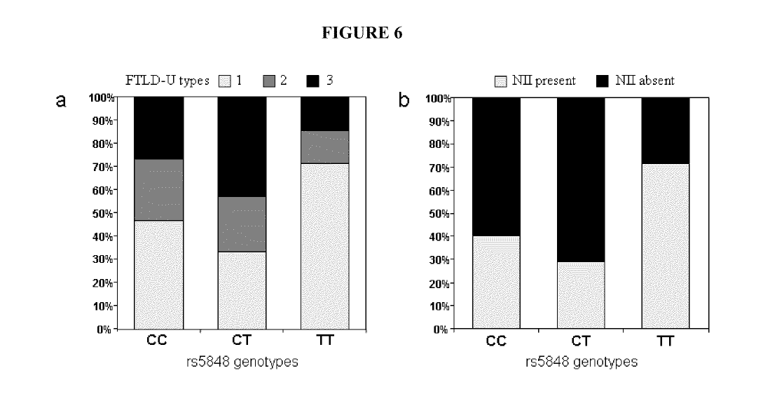

Overall, FTLD-U subtypes could be determined for 50 FTLD-U patients resulting in 24 patients with FTLD-U type 1 (48%), 11 patients with FTLD-U type 2 (22%), and 15 patients with FTLD-U type 3 (30%). For the remaining 3 patients, the FTLD-U subtype could not be unambiguously assigned in part because inclusions were sparse (FTLD-U type 1 versus type 3). Stratification of FTLD-U patients by rs5848 genotype exhibited a non-significant increase in the frequency of FTLD-U type 1 pathology in carriers homozygous for the risk T-allele (71% in TT versus 33% in CT and 47% in CC carriers; p=0.24) (FIG. 6A). Moreover, NIIs were significantly more common in the subgroup of homozygous T-allele carriers compared to heterozygous CT or homozygous CC carriers (71% in TT versus 29% in CT and 40% in CC carriers; p=0.04) (FIG. 6B). Finally, MND pathology was only present in one of the 15 FTLD-U patients homozygous for the risk T-allele (6.6%) compared to five out of 24 heterozygous CT carriers (20.8%) and six out of 21 homozygous C-allele carriers (28.5%).

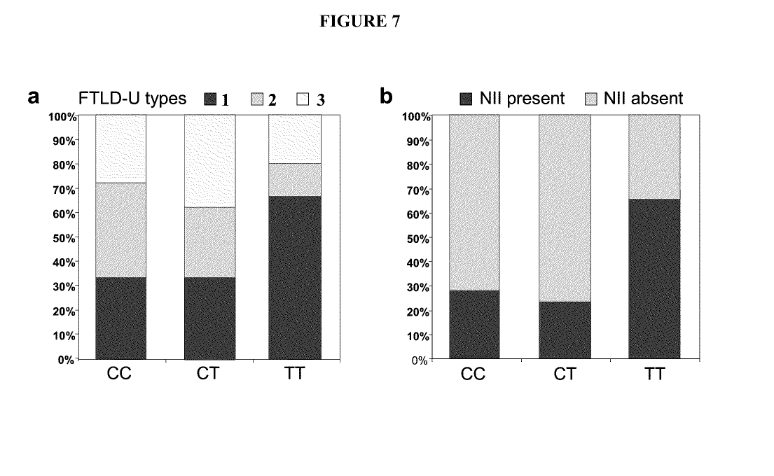

In a second trial, FTLD-U subtypes could be determined for 54 FTLD-U patients resulting in 23 patients with FTLD-U type 1 (42.6%), 15 patients with FTLD-U type 2 (27.8%) and 16 patients with FTLD-U type 3 (29.6%). For the remaining 3 patients, the FTLD-U subtype could not be unambiguously assigned in part because inclusions were sparse (FTLD-U type 1 versus type 3). Stratification of FTLD-U patients by rs5848 genotype exhibited a non-significant increase in the frequency of FTLD-U type 1 pathology (resembling GRN mutation carriers) in TT carriers (66.7% in TT versus 33.3% in CT and CC carriers; p=0.26; Fisher exact test) (FIG. 7A). Moreover, NIIs were significantly more common in the subgroup of TT carriers compared to CT or CC carriers (66.7% in TT versus 23.8% in CT and 27.8% in CC carriers; p=0.02; Fisher exact test) (FIG. 7B). Finally, MND pathology, which is rare or absent in GRN mutation carriers, was only present in one of the 15 FTLD-U patients homozygous for the risk T-allele (6.7%) compared to five out of 23 CT carriers (21.7%) and five out of 21 CC carriers (23.8%).

### Example 2

### rs5848 Modifies TDP43 Pathology in AD Patients and Increases the Risk to Develop Hippocampal Sclerosis

The mutant ‘T’ allele of rs5848 located in the miRNA-659 binding site of GRN is associated with a decreased expression of GRN polypeptide through the augmented suppression of PGRN translation. TDP-43 was recently identified as the major disease protein in the ubiquitinated inclusions observed in the brains of FTLD-U and ALS patients. In addition, TDP-43 immunoreactivity was observed in 20-30% of patients with Alzheimer's disease (AD) and in Lewy-body related diseases defining a novel class of TDP43 proteinopathies.

In this study, genotyping of variant rs5848 in a pathological confirmed series of Alzheimer's disease (AD) patients from the MCJ brain bank was performed. TDP-43 immunostaining was performed in a subset of these patients to determine the presence or absence of TDP-43 pathology. Comparison of allele- and genotype-frequencies using chi-square tests revealed both an allelic and a genotypic association of rs5848 with the development of TDP43 pathology (p_{allelic}=0.02; p_{genotypic}=0.009) (Table 15). In this series AD patients carrying one or two copies of the mutant ‘T’-allele of rs5848 were estimated to have a 2.2 times increased risk of developing TDP-43 pathology in their brain, compared to AD patients homozygous for the wild-type C-allele (Odds ratio (OR)=2.2; 95% confidence interval CI:4.13-1.18).

TABLE 15
rs5848 in AD pathological confirmed cases

| OBS (N) | | | | EXP (N) | | | |
|---|---|---|---|---|---|---|---|
| genotypes rs5848 | TDP (−) | TDP (+) | | genotypes | cases | controls | |
| CC | 52 | 24 | 76 | mm | 43.931 | 32.069 | 76 |
| CT | 43 | 40 | 83 | mv | 47.977 | 35.023 | 83 |
| TT | 5 | 9 | 14 | vv | 8.092 | 5.908 | 14 |
| | 100 | 73 | 173 | | 100 | 73 | 173 |
| OBS (%) | | | | CHI | | | |
| genotypes rs5848 | TDP (−) | TDP (+) | | genotypes | cases | controls | |
| CC | 0.52 | 0.33 | | mm | 1.482215 | 2.030431 | 3.513 |
| CT | 0.43 | 0.55 | | mv | 0.516276 | 0.707228 | 1.224 |
| TT | 0.05 | 0.12 | | vv | 1.181771 | 1.618865 | 2.801 |
| | 1.00 | 1.00 | | | 3.180262 | 4.356524 | 7.537 |
| p (genotypes, 2df) | 0.0230891 | 0.02309 | | | | | |
| OBS (N) | | | | EXP (N) | | | |
| alleles rs5848 | TDP (−) | TDP (+) | | alleles | cases | controls | |
| C | 147 | 88 | 235 | m | 135.8382 | 99.16185 | 235 |
| T | 53 | 58 | 111 | v | 64.16185 | 46.83815 | 111 |
| | 200 | 146 | 346 | | 200 | 146 | 346 |
| OBS (%) | | | | CHI | | | |
| alleles rs5848 | TDP (−) | TDP (+) | | alleles | cases | controls | |
| C | 0.74 | 0.60 | | m | 0.917172 | 1.256399 | 2.174 |
| T | 0.27 | 0.40 | | v | 1.94176 | 2.659945 | 4.602 |
| | 1.00 | 1.00 | | | 2.858931 | 3.916344 | 6.775 |
| p (alleles, 1df) | 0.0092429 | 0.00924 | | | | | |
| Odds Ratio | CC | CT + TT | | | | | |
| TDP− | 52 | 48 | | OR | 2.211805556 | 0.793809 | |
| TDP+ | 24 | 49 | | | 0.319591822 | 1.420209 | 0.167 |
| | 76 | 97 | | | 0.626399971 | | |
| | | | | 95% | 4.137985793 | 1.182238 | |
| | | | | CI | | | |
| | | | | | boven | onder | |

Next, rs5848 allele- and genotype-frequencies were analyzed in the setting of hippocampal sclerosis. AD patients carrying the mutant ‘T’-allele of rs5848 significantly more commonly presented with hippocampal sclerosis as an accompanying disease phenotype. The T-allele frequency increased from 29% in AD patients without hippocampal sclerosis to 45% in patients with hippocampal sclerosis (p=0.001) (Table 16). The OR associated with the development of hippocampal sclerosis for AD patients carrying the minor T-allele of rs5848 compared to homozygous C-allele carriers were estimated to be 2.67 (95% confidence interval (CI): 5.20-1.38).

TABLE 16
rs5848 in AD with and without hippocampal sclerosis
AD patients

| OBS (N) | | | | EXP (N) | | | |
|---|---|---|---|---|---|---|---|
| genotypes | HpScl (+) | HpScl (−) | | genotypes | cases | controls | |
| CC | 13 | 148 | 161 | mm | 23.000 | 138.000 | 161 |
| CT | 32 | 153 | 185 | mv | 26.429 | 158.571 | 185 |
| TT | 8 | 17 | 25 | vv | 3.571 | 21.429 | 25 |
| | 53 | 318 | 371 | | 53 | 318 | 371 |
| OBS (%) | | | | CHI | | | |
| genotypes | HpScl (+) | HpScl (−) | | genotypes | cases | controls | |
| CC | 0.25 | 0.47 | | mm | 4.347826 | 0.724638 | 5.072 |
| CT | 0.60 | 0.48 | | mv | 1.174517 | 0.195753 | 1.37 |
| TT | 0.15 | 0.05 | | vv | 5.491429 | 0.915238 | 6.407 |
| | 1.00 | 1.00 | | | 11.01377 | 1.835629 | 12.85 |
| p (genotypes, 2df) | 0.001621 | 0.00162 | | | | | |
| OBS (N) | | | | EXP (N) | | | |
| alleles | HpScl (+) | HpScl (−) | | alleles | cases | controls | |
| C | 58 | 449 | 507 | m | 72.42857 | 434.5714 | 507 |
| T | 48 | 187 | 235 | v | 33.57143 | 201.4286 | 235 |
| | 106 | 636 | 742 | | 106 | 636 | 742 |
| OBS (%) | | | | CHI | | | |
| alleles | HpScl (+) | HpScl (−) | | alleles | cases | controls | |
| C | 0.55 | 0.71 | | m | 2.874331 | 0.479055 | 3.353 |
| T | 0.45 | 0.29 | | v | 6.201216 | 1.033536 | 7.235 |
| | 1.00 | 1.00 | | | 9.075547 | 1.512591 | 10.59 |
| p (alleles, 1df) | 0.0011382 | 0.00114 | | | | | |
| Odds Ratio | CT + TT | CC | | | | | |
| HpScl (+) | 40 | 13 | | OR | 2.678733 | 0.985344 | |
| HpScl (−) | 170 | 148 | | | 0.3384704 | 1.648746 | 0.322 |
| | 210 | 161 | | | 0.6634019 | | |
| | | | | CI | 5.2004536 | 1.379805 | |
| | | | | | boven | onder | |

Genotyping of variant rs5848 in a pathological confirmed series of Alzheimer's disease (AD) patients from the MCJ brain bank was performed again. TDP-43 immunostaining was performed in a subset of these patients to determine the presence or absence of TDP-43 pathology. Comparison of allele- and genotype frequencies showed a trend towards an increased frequency of the r5848 T-allele in AD patients with TDP-43 pathology, from 29% in AD patients without TDP-43 pathology to 34% in AD patients with TDP-43 pathology, although this finding did not reach significance (p_{allelic}=0.15; p_{genotypic}=0.08) (Table 17). In this series, it was estimated that AD patients carrying one or two copies of the mutant ‘T’-allele of rs5848 have a 1.39 times increased risk of developing TDP-43 pathology in their brain, compared to AD patients homozygous for the wild-type C-allele (Odds ratio (OR)=1.39; 95% confidence interval CI:1.92-1.00; p-value=0.05).

TABLE 17
rs5848 in AD pathological confirmed cases

| OBS (N) | | | | EXP (N) | | | |
|---|---|---|---|---|---|---|---|
| genotypes rs5848 | TDP (−) | TDP (+) | | genotypes | TDP (−) | TDP (+) | |
| CC | 211 | 91 | 302 | CC | 199.301 | 102.699 | 302 |
| CT | 179 | 107 | 286 | CT | 188.742 | 97.258 | 286 |
| TT | 35 | 21 | 56 | TT | 36.957 | 19.043 | 56 |
| | 425 | 219 | 644 | | 425 | 219 | 644 |
| OBS (%) | | | | CHI | | | |
| genotypes rs5848 | TDP (−) | TDP (+) | | genotypes | TDP (−) | TDP (+) | |
| CC | 0.50 | 0.42 | | CC | 0.686704 | 1.332644 | 2.019 |
| CT | 0.42 | 0.49 | | CT | 0.502861 | 0.975872 | 1.479 |
| TT | 0.08 | 0.10 | | TT | 0.103581 | 0.201013 | 0.305 |
| | 1.00 | 1.00 | | | 1.293146 | 2.509529 | 3.803 |
| p (genotypes, 2df) | 0.1493687 | 0.14937 | | | | | |
| OBS (N) | | | | EXP (N) | | | |
| alleles rs5848 | TDP (−) | TDP (+) | | alleles | TDP (−) | TDP (+) | |
| C | 601 | 289 | 890 | C | 587.3447 | 302.6553 | 890 |
| T | 249 | 149 | 398 | T | 262.6553 | 135.3447 | 398 |
| | 850 | 438 | 1288 | | 850 | 438 | 1288 |
| OBS (%) | | | | CHI | | | |
| alleles rs5848 | TDP (−) | TDP (+) | | alleles | TDP (−) | TDP (+) | |
| C | 0.71 | 0.66 | | C | 0.317474 | 0.616102 | 0.934 |
| T | 0.29 | 0.34 | | T | 0.709929 | 1.377717 | 2.088 |
| | 1.00 | 1.00 | | | 1.027403 | 1.993819 | 3.021 |
| p (alleles, 1df) | 0.0821815 | 0.08218 | | | | | |
| Odds Ratio | CC | CT + TT | | | | | |
| TDP− | 211 | 214 | | OR | 1.386874807 | 0.327053 | |
| TDP+ | 91 | 128 | | | 0.167969475 | 0.656273 | −0 |
| | 302 | 342 | | | 0.329220172 | | |
| | | | | 95% CI | 1.927594876 | 0.997835 | |
| p = 0.051175653 | | | | | boven | onder | |

Next, rs5848 allele- and genotype-frequencies were analyzed in the setting of hippocampal sclerosis. AD patients carrying the mutant ‘T’-allele of rs5848 significantly more commonly presented with hippocampal sclerosis as an accompanying disease phenotype. The T-allele frequency increased from 30% in AD patients without hippocampal sclerosis to 43% in patients with hippocampal sclerosis (p=0.003) (Table 18). The OR associated with the development of hippocampal sclerosis for AD patients carrying the minor T-allele of rs5848 compared to homozygous C-allele carriers were 2.43 (95% confidence interval (CI): 4.43-1.33).

TABLE 18
rs5848 in AD with and without hippocampal sclerosis
AD patients

| OBS (N) | | | | EXP (N) | | | |
|---|---|---|---|---|---|---|---|
| genotypes | HpScl (+) | HpScl (−) | | genotypes | HpScl (+) | HpScl (−) | |
| CC | 16 | 286 | 302 | CC | 26.730 | 275.270 | 302 |
| CT | 33 | 253 | 286 | CT | 25.314 | 260.686 | 286 |
| TT | 8 | 48 | 56 | TT | 4.957 | 51.043 | 56 |
| | 57 | 587 | 644 | | 57 | 587 | 644 |
| OBS (%) | | | | CHI | | | |
| genotypes | HpScl (+) | HpScl (−) | | genotypes | HpScl (+) | HpScl (−) | |
| CC | 0.28 | 0.49 | | CC | 4.307134 | 0.41824 | 4.725 |
| CT | 0.58 | 0.43 | | CT | 2.333908 | 0.226632 | 2.561 |
| TT | 0.14 | 0.08 | | TT | 1.868802 | 0.181468 | 2.05 |
| | 1.00 | 1.00 | | | 8.509844 | 0.826339 | 9.336 |
| p (genotypes, 2df) | 0.0093902 | 0.00939 | | | | | |
| OBS (N) | | | | EXP (N) | | | |
| alleles | HpScl (+) | HpScl (−) | | alleles | HpScl (+) | HpScl (−) | |
| C | 65 | 825 | 890 | C | 78.77329 | 811.2267 | 890 |
| T | 49 | 349 | 398 | T | 35.22671 | 362.7733 | 398 |
| | 114 | 1174 | 1288 | | 114 | 1174 | 1288 |
| OBS (%) | | | | CHI | | | |
| alleles | HpScl (+) | HpScl (−) | | alleles | HpScl (+) | HpScl (−) | |
| C | 0.57 | 0.70 | | C | 2.408222 | 0.233848 | 2.642 |
| T | 0.43 | 0.30 | | T | 5.38522 | 0.522926 | 5.908 |
| | 1.00 | 1.00 | | | 7.793442 | 0.756774 | 8.55 |
| p (alleles, 1df) | 0.0034548 | 0.00345 | | | | | |
| Odds Ratio | CT + TT | CC | | | | | |
| HpScl (+) | 41 | 16 | | OR | 2.4348007 | 0.889865 | |
| HpScl (−) | 301 | 286 | | | 0.3061193 | 1.489859 | 0.29 |
| | 342 | 302 | | | 0.5999938 | | |
| | | | | CI | 4.4364684 | 1.336255 | |
| | | | | | boven | onder | |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for diagnosing dementia in a mammal suspected of having dementia, wherein said method comprises determining whether or not said mammal contains a mutant T allele of rs5848, wherein said mammal has dementia if said mammal contains said mutant T allele.

2. The method of claim 1, wherein said mammal is a human.

3. The method of claim 1, wherein said dementia is frontotemporal lobar degeneration.

4. The method of claim 1, wherein said dementia is frontotemporal dementia.

5. The method of claim 1, wherein said method comprises determining whether or not said mammal is heterozygous for said mutant T allele, wherein said mammal has dementia if said mammal is heterozygous for said mutant T allele.

6. The method of claim 1, wherein said method comprises determining whether or not said mammal is homozygous for said mutant T allele, wherein said mammal has dementia if said mammal is homozygous for said mutant T allele.

7. A method for classifying a mammal as being at risk of developing dementia, wherein said method comprises determining whether or not a mammal contains a mutant T allele of rs5848, wherein that said mammal is at risk of developing dementia if said mammal contains said mutant T allele.

8. The method of claim 7, wherein said mammal is a human.

9. The method of claim 7, wherein said dementia is frontotemporal lobar degeneration.

10. The method of claim 7, wherein said dementia is frontotemporal dementia.

11. The method of claim 7, wherein said method comprises determining whether or not said mammal is heterozygous for said mutant T allele, wherein said mammal is at risk of developing dementia if said mammal is heterozygous for said mutant T allele.

12. The method of claim 7, wherein said method comprises determining whether or not said mammal is homozygous for said mutant T allele, wherein said mammal is at risk of developing dementia if said mammal is homozygous for said mutant T allele.
